(19) United States
(12) Patent Application Publication
(10) Pub. No.: US 2015/0359477 A1
(43) Pub. Date: Dec. 17, 2015

(54) SYSTEMS AND METHODS FOR ASSESSING POSSIBLE COGNITIVE IMPAIRMENT

(71) Applicants: Akshitha Ramachandran, Winchester, MA (US); Sara Elizabeth Luettgen, Winchester, MA (US); Alexandra Martirosian, Winchester, MA (US); Kiara Isabel Wahnschafft, Winchester, MA (US); Rebecca Li Hao, Winchester, MA (US); David C. Petty, Cambridge, MA (US); Alexandra Geyer, Winchester, MA (US)

(72) Inventors: Akshitha Ramachandran, Winchester, MA (US); Sara Elizabeth Luettgen, Winchester, MA (US); Alexandra Martirosian, Winchester, MA (US); Kiara Isabel Wahnschafft, Winchester, MA (US); Rebecca Li Hao, Winchester, MA (US); David C. Petty, Cambridge, MA (US); Alexandra Geyer, Winchester, MA (US)

(21) Appl. No.: 14/735,537

(22) Filed: Jun. 10, 2015

Related U.S. Application Data
(60) Provisional application No. 62/010,508, filed on Jun. 11, 2014.

(51) Int. Cl. A61B 5/00; G09B 7/02

(52) U.S. Cl. CPC A61B 5/4064; A61B 5/7246; A61B 5/486; A61B 5/7282; G09B 7/02; A61B 2560/0475

(57) ABSTRACT

The systems and methods described herein relate to impairment assessments that may use frequent baseline examinations of users to generate a performance threshold that allows more reliable impairment tests to be administered at accident sites. Assessment test results may also be forwarded to other parties to indicate when a user should seek medical attention.

### CROSS-REFERENCE TO RELATED APPLICATIONS

This application claims the benefit of U.S. Prov. App. No. 62/010,508 filed on Jun. 11, 2014, the entire contents of which is hereby incorporated by reference.

### TECHNICAL FIELD

The systems and methods described herein relate to assessing possible cognitive impairment, which may include identifying possible concussions.

### BACKGROUND

People who have suffered an initial brain injury and are not properly treated are at high risk of further injury, including potential death from swelling in the brain. Currently, many attempt to identify concussion risk by asking a potentially injured person to “close their eyes and stand” (to test balance) or to describe “how much their head hurts.” But concussion symptoms are often initially subtle. Severe symptoms such as headaches and dizziness often appear only after significant delay. Worse, children at play or athletes in sports may be motivated to downplay symptoms indicative of concussions in order to continue their activity. Consequently, concussed athletes frequently resume playing despite an underlying injury, to their detriment. Improved methods for assessing potential brain injuries are needed.

The occurrence of brain injury is often not initially apparent to an untrained observer. A medical professional can provide accurate diagnoses, but medical professionals are frequently unavailable at likely sites of concussions. Several concussion-testing programs rely on biennial baseline tests, but a 2007 study led by Steven Broglio, director at the NeuroSport Research Laboratory at the University of Michigan, found that even uninjured students showed little consistency in baseline cognitive tests taken a mere 45 days apart. Thus there exists a need in the art for systems and methods for assessing user impairment based on personalized, current cognitive performance baselines.

### BRIEF SUMMARY

In certain aspects, the system described herein assesses possible brain injury in a human. In such aspects, the system comprises a user database configured to store a performance threshold associated with a first user, and a processor operably connected to the user database. The processor is configured to present one or more assessment tasks to the first user, receive input from the first user in response to the one or more assessment tasks, and score the input received in response to the one or more assessment tasks. The one or more assessment tasks may include questions for the first user to answer, actions for the first user to perform, or other suitable tasks. The processor is further configured to output an indicator representative of a comparison of the input score to the performance threshold stored in the user database. In some implementations, the possible brain injury is a possible concussion. In some implementations, one or more elements of the system may be implemented on a cloud computing device.

In some implementations, the processor is further configured to determine whether the first user has completed a baseline test within a predetermined period of time, the baseline test comprising one or more baseline tasks. The predetermined period of time may be one month, three weeks, two weeks, one week, or some other suitable period of time. In some such implementations, the baseline tasks include one or more user status questions (e.g., a symptom question, a fatigue question, a mood question, or other suitable questions), one or more problem-solving tasks (e.g., a Tower of Hanoi task, a mathematical processing task, and a logical reasoning task), one or more attention tasks (e.g., a running memory continuous performance task, and a Stroop test), one or more working memory tasks (e.g., a digit span test, a code substitution task, and a digit set comparison task), one or more reaction time tasks (e.g., a two-choice reaction time test, a four-choice reaction time test, and a procedural reaction time test), one or more visuospatial tasks (e.g., a Trail Making Test, a spatial processing task, and a tracking task), non-cognitive tasks (e.g., a balance task), or other suitable tasks, and the processor may be further configured to randomly generate one or more of such tasks. If the first user has not completed a baseline test within the predetermined period of time, the processor is further configured to output a prompt to request that the first user complete a baseline test and administer a first baseline test comprising one or more baseline tasks to the first user. The performance threshold may be based on input received from the first user in response to the first baseline test. In some such implementations, the processor is further configured to administer the baseline test within a period of time of user exercise in the absence of a possible brain injury. As an illustrative example of such an implementation, the processor may receive a message from a second user (such as a coach or guardian of the first user) indicating when a practice session has been completed, and may respond to the message by prompting the first user to complete a baseline test. The processor may be further configured to administer a second baseline test to the first user and identify a change in performance from the first baseline test to the second baseline test, and in some implementations may modify the performance threshold based on the change in performance. In implementations in which the processor is configured to administer a second baseline test, the processor may be configured to administer the second baseline test to the first user after identifying a possible brain injury.

In some implementations, the processor is further configured to output an indicator representative of the comparison to a second user, who may be a coach, a legal guardian, a referee, an umpire, a trainer, a medical professional, or some other suitable second party. In some such implementations, the processor is further configured to output an indicator representative of the comparison to a third user, to receive feedback from the third user responding to the indicator, and output the third-user feedback to the second user. As an illustrative example of such an implementation, a referee and a medical professional may both receive an indicator representative of the comparison between the input score and the performance threshold, and the referee may further receive the medical professional's comments regarding the comparison.

In some implementations, the processor is further configured to present the one or more assessment tasks for completion within a predetermined period of response time. The predetermined period of response time may be ten minutes, seven minutes, five minutes, four minutes, two minutes, or some other suitable period of response time.

In some implementations, the one or more assessment tasks include one or more user status questions (e.g., a symptom question, a fatigue question, a mood question, or other suitable questions), one or more problem-solving tasks (e.g., a Tower of Hanoi task, a mathematical processing task, and a logical reasoning task), one or more attention tasks (e.g., a running memory continuous performance task, and a Stroop test), one or more working memory tasks (e.g., a digit span test, a code substitution task, and a digit set comparison task), one or more reaction time tasks (e.g., a two-choice reaction time test, a four-choice reaction time test, and a procedural reaction time test), one or more visuospatial tasks (e.g., a Trail Making Test, a spatial processing task, and a tracking task), non-cognitive tasks (e.g., a balance task), or other suitable tasks, and the processor may be further configured to randomly generate one or more of such tasks.

In implementations in which the processor is configured to administer a Stroop test, the processor may be further configured to identify whether the first user is color-blind, the first user's type of color-blindness, and Stroop test questions unaffected by the first user's color-blindness. In some such implementations, the processor is further configured to identify the first user's type of color-blindness based on at least one of the one or more assessment tasks.

In some implementations, the processor is further configured to receive sensor data associated with the user, wherein the input score is based on the sensor data. Such data may include accelerometer data, touchscreen data, heart rate data, or other suitable data.

In some implementations, the processor is further configured to identify a location of and/or contact a medical care facility near the first user.

In some implementations, the one or more assessment tasks include one or more gating tasks associated with severe impairment, and the processor is further configured to administer the one or more gating tasks to the first user, determine whether the input received in response to the one or more gating tasks is indicative of severe impairment, and, if the input received in response to the one or more gating tasks is indicative of severe impairment, stop administration of assessment tasks and output a warning.

In certain aspects, the computer-implemented method described herein assesses possible brain injury in a human. In such aspects, the method comprises presenting one or more assessment tasks to administer to a first user, receiving input from the first user in response to the one or more assessment tasks, scoring the input received in response to the one or more assessment tasks, and identifying a performance threshold associated with the first user. The one or more assessment tasks may include questions for the first user to answer, actions for the first user to perform, or other suitable tasks. The method outputs an indicator representative of a comparison of the input score to the performance threshold. In some implementations, the possible brain injury is a possible concussion.

In some implementations, the method further comprises determining whether the first user has completed a baseline test within a predetermined period of time, the baseline test comprising one or more baseline tasks. The predetermined period of time may be one month, three weeks, two weeks, one week, or some other suitable period of time. In some such implementations, the baseline tasks include one or more user status questions (e.g., a symptom question, a fatigue question, a mood question, or other suitable questions), one or more problem-solving tasks (e.g., a Tower of Hanoi task, a mathematical processing task, and a logical reasoning task), one or more attention tasks (e.g., a running memory continuous performance task, and a Stroop test), one or more working memory tasks (e.g., a digit span test, a code substitution task, and a digit set comparison task), one or more reaction time tasks (e.g., a two-choice reaction time test, a four-choice reaction time test, and a procedural reaction time test), one or more visuospatial tasks (e.g., a Trail Making Test, a spatial processing task, and a tracking task), non-cognitive tasks (e.g., a balance task), or other suitable tasks, and the method may further comprise randomly generating one or more of such tasks. If the first user has not completed a baseline test within the predetermined period of time, the method outputs a prompt to request that the first user complete a baseline test and administers a first baseline test comprising one or more baseline tasks to the user. The performance threshold may be based on input received from the first user in response to the first baseline test. In some such implementations, the method administers the baseline test within a period of time of user exercise in the absence of a possible brain injury. As an illustrative example of such an implementation, the method may prompt the first user to complete a baseline test when a second user (such as a coach or guardian of the first user) indicates that a practice session has been completed. The method may further administer a second baseline test to the first user and identify a change in performance from the first baseline test to the second baseline test, and in some implementations may modify the performance threshold based on the change in performance. In implementations in which the method administers a second baseline test, the method may administer the second baseline test to the first user after identifying a possible brain injury.

In some implementations, the method further comprises outputting an indicator representative of the comparison to a second user, who may be a coach, a legal guardian, a referee, an umpire, a trainer, a medical professional, or some other suitable second party. In some such implementations, the method further comprises outputting an indicator representative of the comparison to a third user, receiving feedback from the third user responding to the indicator, and outputting the third-user feedback to the second user. As an illustrative example of such an implementation, a referee and a medical professional may both receive an indicator representative of the comparison between the input score and the performance threshold, and the referee may further receive the medical professional's comments regarding the comparison.

In some implementations, the method further comprises presenting the one or more assessment tasks for completion within a predetermined period of response time. The predetermined period of response time may be ten minutes, seven minutes, five minutes, four minutes, two minutes, or some other suitable period of response time.

In some implementations, the one or more assessment tasks include one or more user status questions (e.g., a symptom question, a fatigue question, a mood question, or other suitable questions), one or more problem-solving tasks (e.g., a Tower of Hanoi task, a mathematical processing task, and a logical reasoning task), one or more attention tasks (e.g., a running memory continuous performance task, and a Stroop test), one or more working memory tasks (e.g., a digit span test, a code substitution task, and a digit set comparison task), one or more reaction time tasks (e.g., a two-choice reaction time test, a four-choice reaction time test, and a procedural reaction time test), one or more visuospatial tasks (e.g., a Trail Making Test, a spatial processing task, and a tracking task), non-cognitive tasks (e.g., a balance task), or other suitable tasks, and the method may further comprise randomly generating one or more of such tasks.

In implementations in which the method comprises administering a Stroop test, the method may further comprise identifying whether the first user is color-blind, the first user's type of color-blindness, and Stroop test questions unaffected by the first user's color-blindness. In some such implementations, method further comprises identifying the first user's type of color-blindness based on at least one of the one or more assessment tasks.

In some implementations, the method further comprises receiving sensor data associated with the user, wherein the input score is based on the sensor data. Such data may include accelerometer data, touchscreen data, heart rate data, or other suitable data.

In some implementations, the method further comprises identifying a location of a medical care facility near the first user.

In some implementations, the one or more assessment tasks include one or more gating tasks associated with severe impairment, and the method further comprises administering the one or more gating tasks to the first user, determining whether the input received in response to the one or more gating tasks is associated with severe impairment, and, if the input received in response to the one or more gating tasks is associated with severe impairment, stopping administration of assessment tasks and outputting a warning.

### BRIEF DESCRIPTION OF THE DRAWINGS

The systems and methods described herein are set forth in the appended list of claims. However, for the purpose of explanation, several implementations are set forth in the following drawings.

FIG. 1 is a block diagram of an injury assessment system, according to an illustrative implementation;

FIG. 2 is a block diagram of a user device, according to an illustrative implementation;

FIG. 3 is a block diagram of an assessment server, according to an illustrative implementation;

FIG. 4 is a flow chart of an injury assessment process, according to an illustrative implementation;

FIG. 5 is a flow chart of a test reminder process, according to an illustrative implementation;

FIG. 6 is a flow chart of a baseline measurement process, according to an illustrative implementation;

FIG. 7 is a screenshot of a test selection screen, according to an illustrative implementation;

FIG. 8 is a screenshot of a baseline test instruction screen, according to an illustrative implementation;

FIG. 9 is a screenshot of an orientation question instruction screen, according to an illustrative implementation;

FIG. 10 is a screenshot of an orientation question screen, according to an illustrative implementation;

FIG. 11 is a screenshot of a symptom question instruction screen, according to an illustrative implementation;

FIG. 12 is a screenshot of a symptom question screen, according to an illustrative implementation;

FIG. 13 is a screenshot of a Trail Making Test instruction screen, according to an illustrative implementation;

FIG. 14 is a screenshot of a Trail Making Test screen, according to an illustrative implementation;

FIG. 15 is a screenshot of a first word recall test instruction screen, according to an illustrative implementation;

FIG. 16 is a screenshot of a first word recall test screen, according to an illustrative implementation;

FIG. 17 is a screenshot of a Stroop test instruction screen, according to an illustrative implementation;

FIG. 18 is a screenshot of a Stroop test screen, according to an illustrative implementation;

FIG. 19 is a screenshot of a second word recall test instruction screen, according to an illustrative implementation;

FIG. 20 is a screenshot of a second word recall test screen, according to an illustrative implementation;

FIG. 21 is a screenshot of a digit span test instruction screen, according to an illustrative implementation;

FIG. 22 is a screenshot of a digit span test screen, according to an illustrative implementation;

FIG. 23 is a screenshot of an assessment test screen, according to an illustrative implementation;

FIG. 24 is a screenshot of an assessment test screen, according to an illustrative implementation;

FIG. 25 is a screenshot of a second party welcome screen, according to an illustrative implementation;

FIG. 26 is a screenshot of an athlete test review screen, according to an illustrative implementation;

FIG. 27 is a screenshot of a group baseline review screen, according to an illustrative implementation; and

FIG. 28 is a block diagram of a computing device for performing any of the processes described herein, according to an illustrative implementation.

### DETAILED DESCRIPTION

In the following description, numerous details are set forth for the purpose of explanation. However, one of ordinary skill in the art will realize that the implementations described herein may be practiced without the use of these specific details and that the implementations described herein may be modified, supplemented, or otherwise altered without departing from the scope of the systems and methods described herein.

The systems and methods described herein relate to assessing possible brain injuries or impairment. A user is associated with a performance threshold, which may be based on regular baseline tests administered to the user. One or more assessment tasks are administered to the user, and input received from the user in response to the assessment tasks is scored. An indicator representative of a comparison of the input score to the performance threshold is outputted-which may include providing the comparison to another user, such as a coach, a referee, a parent, a legal guardian, a medical professional, or some other suitable user-indicating whether the user is likely to be suffering impairment.

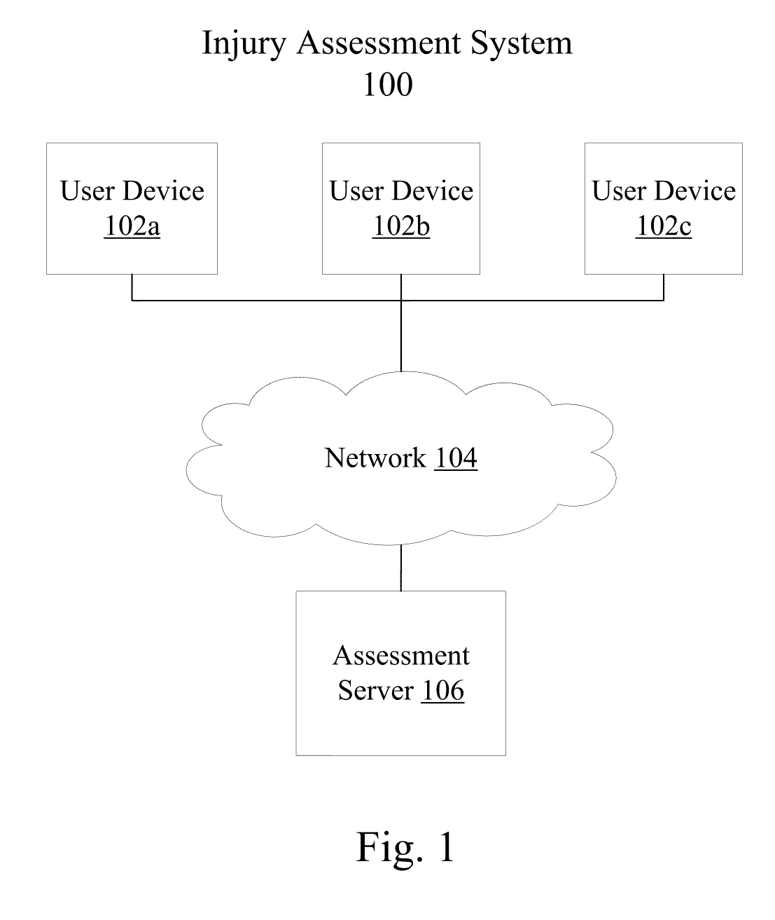

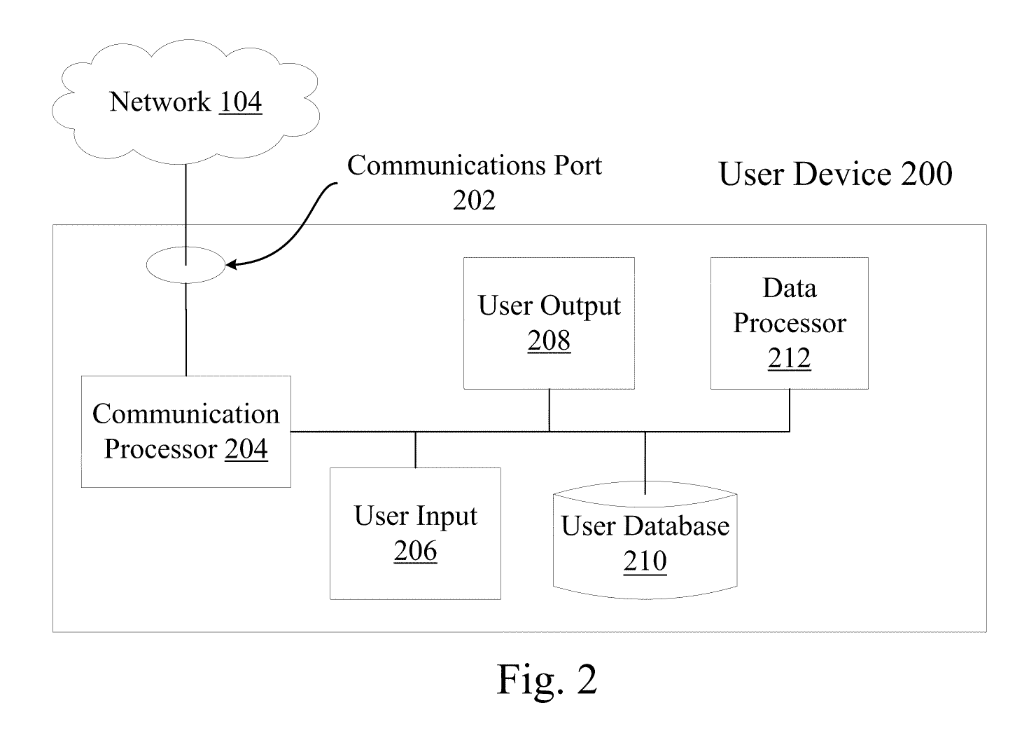

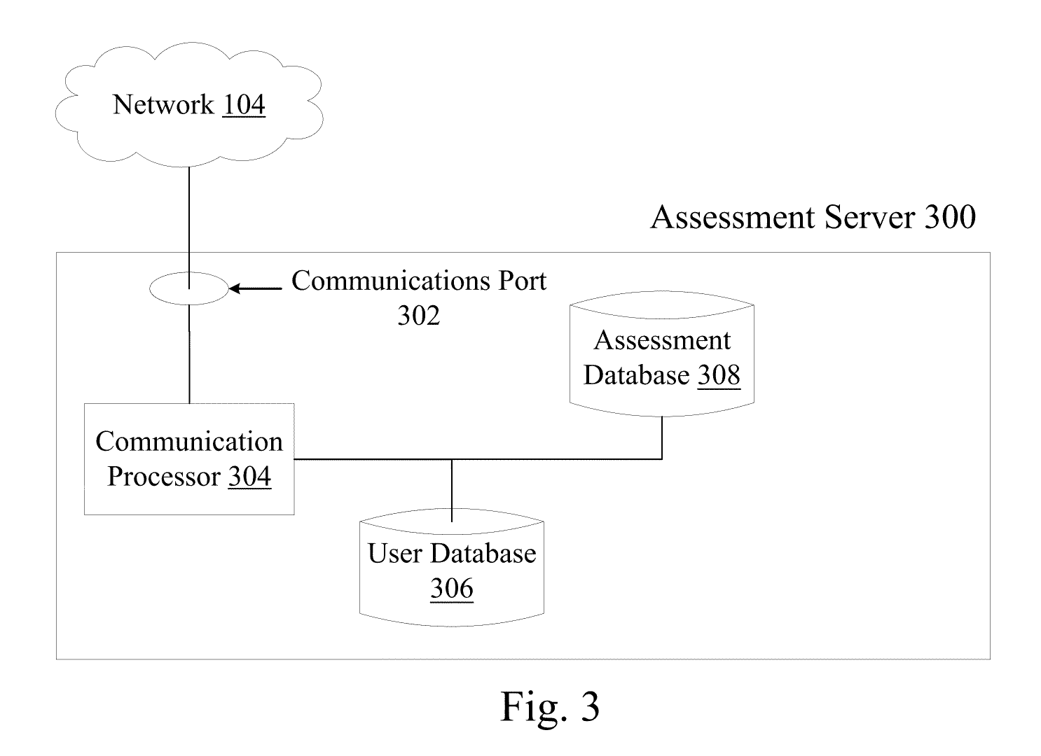

FIG. 1 is an illustrative block diagram of an injury assessment system 100, which assesses whether a user is likely to have suffered a brain injury. As depicted, three different users interact with injury assessment system 100 through, respectively, user devices 102a-102c (collectively user devices 102), which are described in more detail in relation to FIG. 2. For illustrative purposes, user device 102a is associated with a first user being tested, while user devices 102b and 102c are associated with receiving users associated with the first user, such as coaches, legal guardians, referees, umpires, trainers, medical professionals, or other suitable users. User device 102a may administer baseline and assessment tests to the first user, and user devices 102b and 102c may provide information regarding the first user's results to individuals authorized to receive such information. There may be a different number of user devices 102 than are depicted here, and there may be a different number of tested users and receiving users. Information regarding user baseline and assessment tests may be transmitted to and from user devices 102 through network 104. Network 104 is a computer network, and in certain implementations may be the Internet. User information may be stored in assessment server 106, which may be a server, a personal computer, a mainframe, a cluster of computing devices, or some other suitable computing device, and is described in more detail in relation to FIG. 3. In some implementations, user information is stored entirely on one or more user devices 102, and there is no assessment server 106. In such implementations, calculation of a user's performance threshold and analysis of a user's performance relative to such a threshold may be performed by one or more user devices 102.

FIG. 2 is an illustrative block diagram of a user device 200, which may be a smartphone, a tablet computer, a personal laptop computer, a wearable computing device, or some other suitable computing device. User device 200 may act as one of the user devices 102 of FIG. 1. User device 200 may administer tests to assess possible brain injury in a user and display information regarding one or more user's tests. User device 200 transmits and receives data through communication port 202. Communications processor 204 may send and receive information regarding user tests through communication port 202. User input 206 may receive user responses to tasks, requests for information regarding a user, or other suitable information; user output 208 may present assessment tasks, baseline tasks, assessments of impairment, or other suitable information. User database 210 stores information about a tested user, which may include the user's baseline test results, the user's threshold performance, which tested users' data each receiving user is permitted to view, tasks to administer to the user, or other suitable information. Data processor 212 may identify tasks to administer to a user, score user responses to tasks, output information related to a user's performance threshold, output information regarding an impairment assessment of a user, identify and/or contact a medical facility close to the user, or perform other suitable actions.

The depicted communication port 202 is a network port which sends and receives baseline and assessment information via network 104. Baseline information may include one or more of a user's performance threshold, when a user last completed a baseline test, when the user is due for another baseline test, a user's answers to baseline tasks, baseline tasks, rules for generating baseline tasks, or other suitable information. Assessment information may include one or more of a user's performance threshold, assessment tasks, a user's response to assessment tasks, rules for generating assessment tasks, feedback regarding an assessment of a user's impairment, or other suitable information. Communication port 202 may include a 100BASE-TX port, a 1000BASE-T port, a 10 GBASE-T port, a WI-FI antenna, a BLUETOOTH antenna, a cellular antenna, or any other suitable network port. In certain implementations, there may be a different number of ports than are depicted. In certain implementations, communication port 202 may provide secure communications, such as by using the Secure Sockets Layer (SSL) protocol, the Transport Layer Security (TLS) protocol, or other suitable protocol.

Communication processor 204 may be a computer processor that sends and receives information via communications port 202. In response to instructions received from data processor 212, communication processor 204 may request baseline or assessment tasks, transmit updates regarding a user's performance threshold or task responses, request information from a website or application server, or transmit other suitable data. Communication processor 204 may further forward or act on information received through communications port 202, such as by recording user baseline information in user database 210, providing information regarding an assessment of a possible brain injury to user output 208 or data processor 212, or taking some other suitable measure. Communication processor 204 may also send and receive user information: as an illustrative example, if an assessment indicates that a user may have suffered a concussion, communication processor 204 may receive GPS information identifying the user's location and transmit a message to a medical professional authorized to view the user's information.

User input 206 may include one or more user input devices, such as a keyboard, a microphone, a touchpad, a touchscreen, an accelerometer, or other suitable input devices. User input 206 allows a user to request administration of a baseline or an assessment test, to respond to baseline and assessment tasks, to request information regarding a user's performance on baseline tests, to request directions to a medical service provider, or to otherwise interact with user device 200. Information received through user input 206 may be transmitted to data processor 212 for processing. In some implementations, a user input device comprising user input 206 may be remote from user device 200, and may transmit information to user device 200 through a USB connection, a BLUETOOTH connection, or some other suitable connection. In some such implementations, user input 206 includes a helmet-mounted sensor (e.g., an accelerometer) that transmits impact data.

User output 208 may include one or more user output devices, such as a display, a touchscreen, speakers, a haptic output, or other suitable output devices. User output 208 may display the output of communication processor 204 and data processor 212 in a user-understandable fashion, which may include presenting baseline tasks or assessment tasks, presenting the results of an assessment test, presenting changes in a user's baseline test results over time, indicating which associated users' information the user may view, or providing some other suitable output.

User database 210 may be a computer-readable and -writable medium storing information about a user, which may include which other users' data the user is authorized to access, the user's performance threshold, baseline tasks, assessment tasks, information regarding appropriate tasks for the user (e.g., whether the user is color-blind and, if so, the user's type of color-blindness), information identifying other users associated with the user, or other suitable information. In some implementations, a portion or the whole of user database 210 may be accessed through a remote network, and may be stored on assessment server 106 or on some other suitable cloud computing drive.

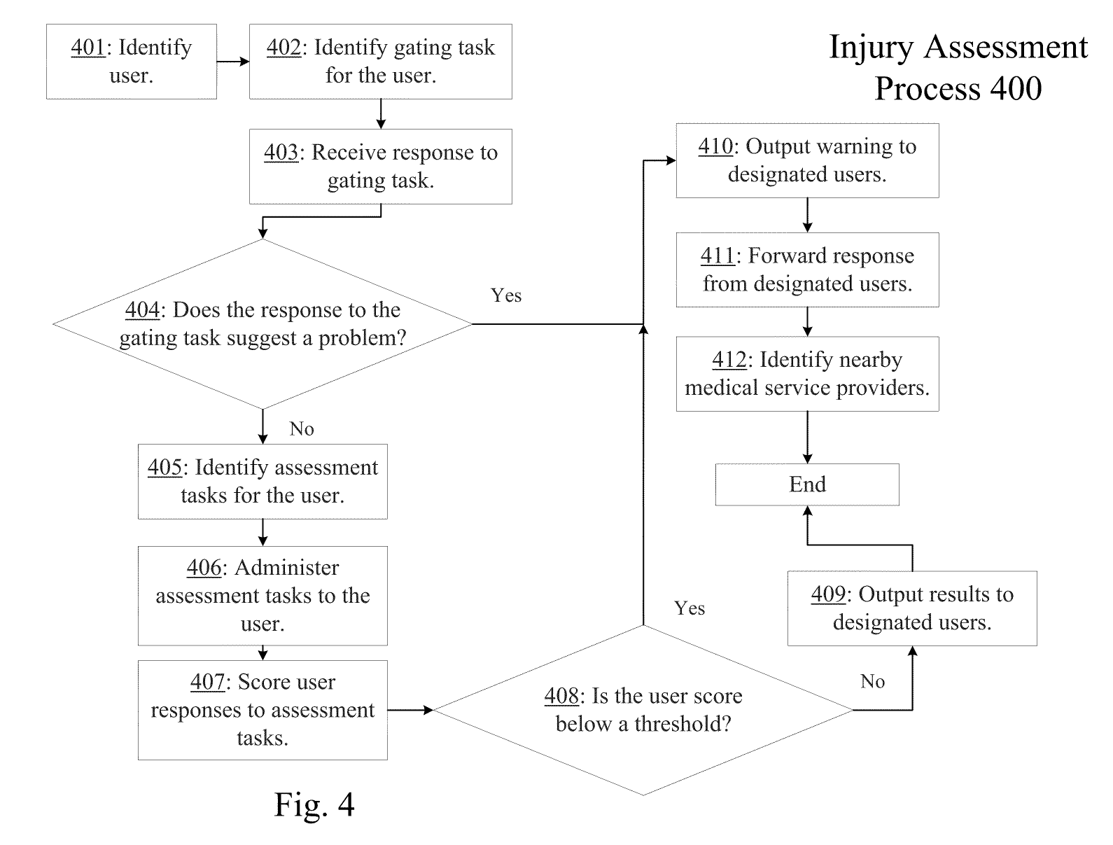

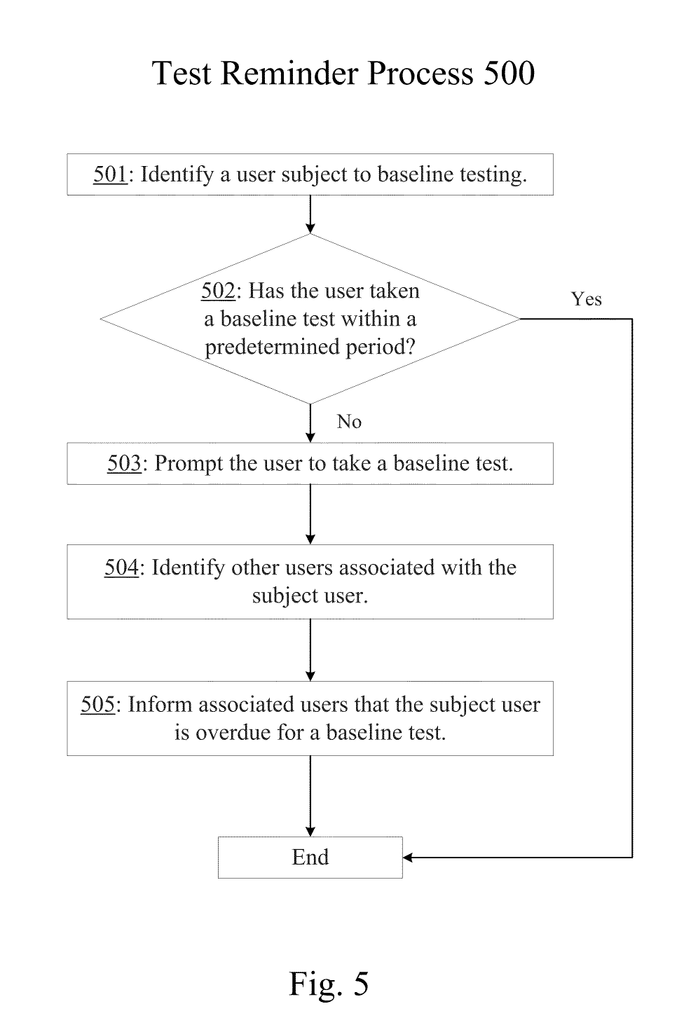

Data processor 212 may be a computer processor that may determine what tasks to present a user, prompt a user to take a baseline test, assess whether a user may be impaired, identify other users to transmit user information to, identify medical facilities in the vicinity of the user, and perform other functions of the user device 200. As described in relation to FIGS. 4 and 5, when data processor 212 recognizes input received from user input 206 as a user request for a baseline or assessment test, data processor 212 identifies tasks for the user based on information in user database 210 or requested, via communication processor 204, from assessment server 106. Data processor 212 administers the identified tasks to the user via user output 208, and, based on the user's responses, may revise the user performance threshold stored in user database 210, output a warning to user output 208, instruct communication processor 204 to transmit a warning to designated related users, or otherwise respond to the user's input.

In some implementations, data processor 212 retrieves and stores information from user database 210, and communication processor 204 synchronizes information between assessment server 106 and user database 210. In some such implementations, communication processor 204 may synchronize information on a periodic basis (e.g., daily, weekly, or monthly), in response to predetermined user actions (e.g., completion of a baseline test), in response to predetermined conditions (e.g., reestablishment of a connection of user device 200 to network 104), or based on some other suitable criteria. As an illustrative example of such an implementation, if no network access is available to user device 200, data processor 212 may identify tasks based on information recorded in user database 210, may cache changes to the user's performance threshold in user database 210 until the user device 200 can reconnect to network 104, and may instruct communication processor 204 to update the user's information on assessment server 106 based on the cached changes once communication processor 204 is able to connect to assessment server 106.

User device 200 administers baseline and assessment tests to users, compares assessment test results with performance thresholds based on baseline tests, and outputs warnings when an assessment indicates impairment. Data processor 212 may prompt a user to take a baseline test via user output 208 after the lapse of a period of time without the user taking a baseline test, based on a message received from another user device 200 (such as a coach indicating that a practice session has ended and all players should take a baseline test) via communication port 202, or based on some other suitable criteria. Data processor 212 administers a baseline test in response to a user requesting a baseline test through user input 206, and may score the baseline test and revise a performance threshold for the user stored in user database 210 based on the user's responses received via user input 206. When a user requests an assessment test, data processor 212 identifies assessment tasks based on information received from assessment server 106 or retrieved from user database 210, administers the tasks through user output 208, and scores responses received through user input 206. If the scored assessment test suggests that the user may be suffering from impairment, data processor 212 informs the user through user output 208 and may instruct communication processor 204 to inform other users listed in user database 210.

FIG. 3 is an illustrative block diagram of an assessment server 300, which may be a server, a personal computer, a mainframe, a cluster of computing devices, or some other suitable computing device. Referring to FIG. 1, assessment server 300 may correspond to assessment server 106. Assessment server 300 may track a user's baseline and assessment task responses and performance threshold, verify whether a user is authorized to view another user's information, identify whether a user is likely to be suffering from an impairment, or perform other suitable tasks. Assessment server 300 transmits and receives data through communications port 302. Communication processor 304 may send and receive information regarding tasks, user responses, and injury assessments through communication port 302. User database 306 stores information about users, which may include information identifying a device associated with a user, a performance threshold of a user, whether a user is colorblind, whether a user is authorized to access another user's information, who has permission to authorize a user to view another user's information, which users should be notified if a user is assessed as having a possible concussion, or other suitable information. Assessment database 308 stores information regarding baseline and assessment tasks, which may include which may include instructions for generating randomized tasks, tasks with model responses and instructions for scoring responses, or other suitable information.

The depicted communication port 302 is a network port which sends and receives assessment information via network 104. Assessment information may include baseline tasks, assessment tasks, user responses to tasks, performance thresholds, assessments of possible brain injury, reminders to take baseline tests, or other suitable information. Communication port 302 may include a 100BASE-TX port, a 1000BASE-T port, a 10 GBASE-T port, a WI-FI antenna, a BLUETOOTH antenna, a cellular antenna, or any other suitable network port. In certain implementations, there may be a different number of ports than are depicted. In certain implementations, communication port 302 may provide secure communications, such as by using the Secure Sockets Layer (SSL) protocol, the Transport Layer Security (TLS) protocol, or other suitable protocol.

Communication processor 304 may be a computer processor that sends and receives information via communications port 302. Communication processor 304 may transmit information regarding a user's records to a user device 102, transmit a reminder to a user device 102 to take a baseline test, transmit tasks or instructions regarding tasks from assessment database 308 to a user device 102, information regarding a user's performance on a baseline or an assessment test to one or more other designated users, or perform other suitable tasks. Such transmissions may be made in response to a request from a user device 102, or may be pushed to user device 102 under certain pre-determined conditions, such as a different user device 102 requesting that a user take a baseline test, the elapse of a predetermined amount of time, or some other suitable condition. As an illustrative example, if a user takes an assessment test and scores below the user's performance threshold, communications port 302 may push a warning regarding the user and a link to a server identifying nearby medical facilities to other user devices 102. Communication processor 304 may also revise information in user database 306 and assessment database 308, such as by updating user's records in user database 306 based on information received from a user device 102 or adding assessment tasks to an assessment database 308 in response to a command from an authorized system administrator.

User database 306 may be a computer-readable and -writable medium storing information about a user, which may include one or more of the user's performance threshold, user devices 102 associated with the user, baseline test result history, assessment test result history, a user's disabilities that are relevant to baseline or assessment tasks, emergency contact information associated with a user, which other users are authorized to view the user's information, which receiving users should receive the user's information and under which circumstances, or other suitable information. As an illustrative example, a coach may designate when a game is taking place, and a referee may be designated as a recipient of an athlete's assessment test results during the game period if the referee is within a predetermined distance from the user.

Assessment database 308 may be a computer-readable and -writable medium storing information regarding baseline and assessment tasks, which may include instructions to display to a user, rules for grading user responses, or other suitable information. As illustrative examples, assessment database 308 may include: computer instructions for generating cognitive tasks, such as a Stroop test, a Trail Making Test, a digit span test, or other suitable tasks; computer instructions for a balance test, including identifying results based on an output of an accelerometer, computer instructions for generating orientation tasks, such as questions to determine whether the user knows what day of the week it is; or other suitable information. In some implementations, user database 306 may be partially or wholly combined with assessment database 308.

Assessment server 300 may record a user's baseline and assessment task responses and performance threshold, determine whether a user may be impaired, and inform other users that a user may be impaired. Communication processor 304 may send and receive information regarding tasks, baseline test results, injury assessments, and other suitable information via communications port 302, and store or retrieve such information from user database 306 and assessment database 308 as appropriate.

FIG. 4 is an illustrative flow chart of an injury assessment process 400. Injury assessment process 400 assesses whether a user is likely to be suffering from cognitive impairment, and generates a warning to the user and/or associated receiving users if so. Referring to FIG. 2, injury assessment process 400 begins with step 401, in which data processor 212 identifies a user requesting an assessment test through user input 206. Data processor 212 may identify the user based on whether the user is the only user associated with the user device 200. In some implementations, a user may request an assessment test on behalf of another user: as an illustrative example, a coach may request an assessment test using her own user device 200, and then hand the user device 200 to one of her players for assessment. In such implementations, the coach may identify the user to data processor 212.

In step 402, data processor 212 identifies a gating task for the user. A gating task may determine whether the user being tested is healthy enough to complete more cognitively challenging tasks, and may include asking the user to answer an orientation question (e.g., the day of the week, the month, the city in which the user lives, or other suitable questions), asking the user to answer a symptom question (e.g., whether the user is nauseous, is experiencing tinnitus, or other suitable questions), asking the user to perform a task, such as holding user device 200 steady for a period of time, or some other suitable task. In step 403, the user provides a response to the gating task through user input 206, and in step 404 data processor 212 determines whether the user response indicates a problem. In some implementations, there may be more than one triage task, in which case steps 402-404 are repeated until the user shows behavior consistent with impairment or provides a positive/normal response to all of the triage tasks. If the user shows impairment, process 400 continues to step 410, described below; otherwise it continues to step 405.

In step 405, data processor 212 identifies one or more assessment tasks for the user. Assessment tasks may include cognitive tests, such as a Stroop test, a Trail Making Test, a digit span test, or other suitable cognitive tests; a symptom question, such as whether the user is experiencing a ringing sensation or feels nauseous; physical activities, such as facing a camera comprising part of user input 206 and looking in a series of prescribed directions; or other suitable tasks. In implementations where assessment tasks include a Stroop test, data processor 212 may determine whether the user is colorblind based on information stored in user database 210, and may administer Stroop test questions in accord with the user's colorblindness or lack thereof. Data processor 212 may retrieve assessment tasks or instructions for generating assessment tasks from user database 210 or, referring to FIG. 3, request them from assessment server 300. In step 406, data processor 212 administers the identified tasks to the user via user output 208. In some implementations, the tasks selected in step 405 are chosen such that the tasks would normally be completed by the identified user within a predetermined period of response time. In some implementations, injury assessment process 400 provides assessment tasks until a predetermined period of response time has lapsed, and thus repeats steps 405-406 until the predetermined period of response time has lapsed. The predetermined period of response time may be two minutes, four minutes, five minutes, six minutes, seven minutes, or some other suitable period of time. In some implementations the predetermined period of response time may be a function of settings created by a trainer, a guardian, or a medical professional, and may vary between users.

In step 407, data processor 212 scores the user responses received via user input 206. In some implementations, user responses to assessment tasks are scored based in part on the rapidity with which the user completed the task, the steadiness with which the user held user device 200, or other suitable criteria. In step 408, data processor 212 compares the user score to a performance threshold associated with the user, which may be stored in user database 210 or requested from assessment server 300. If the user's score meets or exceeds the user's performance threshold, or is outside a predetermined (normal) range around the user's performance threshold, process 400 ends with step 409, in which data processor 212 outputs the results to designated receiving users. Step 409 may include one or more of displaying the result through user output 208, instructing communication processor 204 to transmit the results to assessment server 300 for distribution or directly to other user devices 200 associated with the receiving user, or other suitable actions.

If data processor 212 finds that the user may be suffering from impairment or injury in steps 404 or 408, e.g., because the user's score is outside an acceptable range around the user's performance, process 400 proceeds to step 410, in which a warning is output to designated receiving users. Step 410 may include displaying the result through user output 208, instructing communication processor 204 to transmit the results to assessment server 300 for distribution or directly to other user devices 200 associated with the receiving user, or other suitable actions. In step 411, process 400 may forward responses from one or more designated receiving users to other users, e.g., the tested user and/or another receiving user (such as a coach or referee). As an illustrative example, if a medical professional judges that the results are worrisome but do not constitute an emergency, the medical professional may transmit a message to a coach saying that the player should be taken out of the game but does not need to be taken to a hospital. A response in step 411 may be transmitted from a designated receiving user's user device 200 to assessment server 300 for distribution, or may be directly transmitted to another designated receiving user's user device 200. Process 400 then ends with step 412, in which data processor 212 identifies nearby medical service providers. Data processor 212 may complete step 412 by instructing communication processor 204 to transmit location information to assessment server 300 or to a third-party location server and request location information of nearby medical service providers.

In some implementations, process 400 may exclude one or more of the depicted steps or may perform one or more of the depicted steps in an alternate order. As illustrative examples, process 400 may exclude step 411, may reverse the order of steps 411 and 412, or may only complete step 412 if requested by a user. In some implementations, one or more steps of injury assessment process 400 may be completed by communication processor 304 based on information received from a user device 200.

FIG. 5 is an illustrative flow chart of a test reminder process 500. Referring to FIG. 4, test reminder process 500 prompts users to take the baseline tests that serve as the basis for assessing whether a user may be impaired or injured in process 400. Referring to FIG. 3, test reminder process 500 begins with step 501, in which communication processor 304 identifies a user subject to baseline testing. In step 502, communication processor 304 determines whether the user has taken a baseline test within a predetermined period, which may include retrieving the date of the user's last baseline test from user database 306 or, referring to FIG. 2, requesting the date of the user's last baseline test from the user's user device 200. If the user has taken a baseline test within the predetermined period, test reminder process 500 concludes; if not, it continues to step 503.

In some implementations, the predetermined period of step 502 may be based on when the user last took a baseline test, and the predetermined period may be a period of days, one week, two weeks, three weeks, a month, or some other suitable period of time. In some implementations of step 502, the predetermined period may be based on receipt of a user request that associated users complete a baseline test, and the predetermined period may be a period of ten minutes, a half-hour, an hour, one-and-a-half hours, two hours, or some other suitable period of time. As an illustrative example of the latter implementations, a coach may transmit a message requesting that players complete a baseline test to assessment server 300 during or immediately after a practice session in order for athletes' baselines to be measured under physically active conditions.

In step 503, communication processor 304 transmits a message prompting the user identified in step 501 to the user's device 200. The message may comprise a text message, an app notification, or some other suitable prompt. In step 504, communication processor 304 identifies receiving users associated with the user of step 501 based on information in user database 306. Such other users may include a coach, a parent, a legal guardian, a medical professional, a referee of an upcoming game, or other suitable users. In step 505, communication processor 304 informs the receiving users identified in step 504 that the tested user is overdue for a baseline test. In some implementations, process 500 may exclude one or more of the depicted steps or may perform one or more of the depicted steps in an alternate order. As illustrative examples, steps 503-505 may be completed in a different order or in parallel; steps 504 and 505 may only be completed if the tested user does not begin taking a baseline test within a predetermined period of time after step 503 is completed; or steps 504 and 505 may be excluded entirely.

Referring to FIG. 2, in some implementations, one or more steps of test reminder process 500 may be completed by user device 200. As illustrative examples, data processor 212 may identify whether the user has taken a baseline test based on information stored in user database 210 or received from assessment server 300; data processor 212 may prompt the user to take a baseline test in step 503; and communication processor 204 may transmit messages to complete step 505.

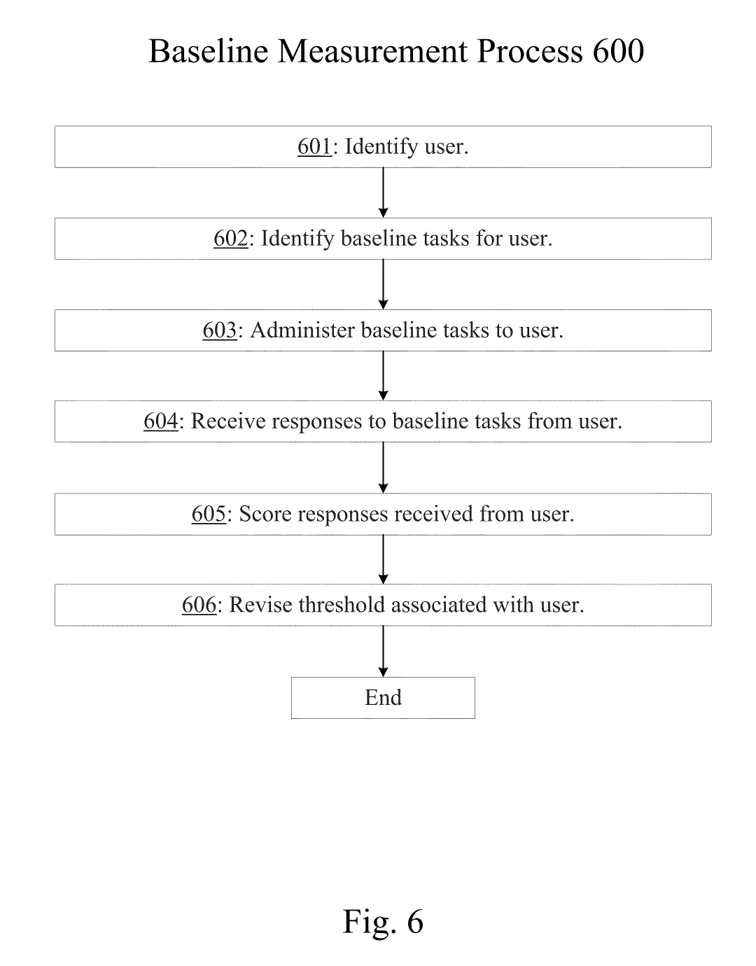

FIG. 6 is an illustrative flow chart of a baseline measurement process 600. Referring to FIG. 4, baseline measurement process 600 determines a performance threshold that serves as a basis for assessing whether a user may be impaired or injured in process 400. Referring to FIG. 2, baseline measurement process 600 begins with step 601, in which data processor 212 identifies the user taking a baseline test. In step 602, data processor 212 identifies baseline tasks for the user, which may include one or more of retrieving baseline tasks from user database 210, requesting baseline tasks from assessment server 300, and generating baseline tasks based on instructions stored in user database 210 or by assessment server 300. Baseline tasks may include a Stroop test, a Trail Making Test, a digit span test, or other suitable cognitive tests; a symptom question, such as whether the user is experiencing a ringing sensation or feels nauseous; physical activities, such as facing a camera comprising part of user input 206 and looking in a series of prescribed directions (which may be indicated by visual or audio instructions); or other suitable tasks. In implementations where baseline tasks include a Stroop test, data processor 212 may determine whether the user is colorblind based on information stored in user database 210, and may identify Stroop test tasks in accord with the user's colorblindness or lack thereof. In step 603, data processor 212 administers the baseline tasks to the user via user output 208; in step 604, data processor 212 receives responses to the baseline tasks from the user via user input 206. Steps 603 and 604 may be repeated until all the tasks have been completed, until a predetermined period of time has elapsed, or based on some other suitable criteria.

In step 606, data processor 212 scores the responses received from the user in step 604. Responses may be scored based on accuracy, speed, any available physical data (e.g., input from a heartbeat monitor, input from an impact sensor, steadiness of the user device 200 as recorded by an accelerometer, or other suitable information), or other suitable information. In step 606, data processor 212 revises a performance threshold associated with the user based on the score calculated in step 605, which may include replacing the performance threshold with a predetermined fraction of the score, recalculating the performance threshold as a weighted average of the newly calculated score and previous scores or the previous threshold, or some other suitable revision. In some implementations, revision of the threshold may also vary based on whether the baseline test was taken within a predetermined amount of time of the conclusion of a practice session: in such implementations, the conclusion of a practice session may be determined based on a message from a designated user (such as a coach), a scheduled time, or based on other suitable criteria. Data processor 212 may store the revised performance threshold in one or both of user database 210 and on assessment server 300. In some implementations, data processor 212 may also display baseline results using user output 208 and may forward results to one or more other designated users via communication processor 204.

In some implementations, one or more of the steps of process 600 may be completed by assessment server 300. As an illustrative example, communication processor 204 may forward the score calculated in step 605 to server 300, which may revise the threshold associated with the user accordingly.

In some implementations, process 600 may exclude one or more of the depicted steps or may perform one or more of the depicted steps in an alternate order. As an illustrative example, process 600 may repeat steps 602-605 such that, after a first baseline task is identified, each subsequent baseline task is identified based on the score calculated in step 605.

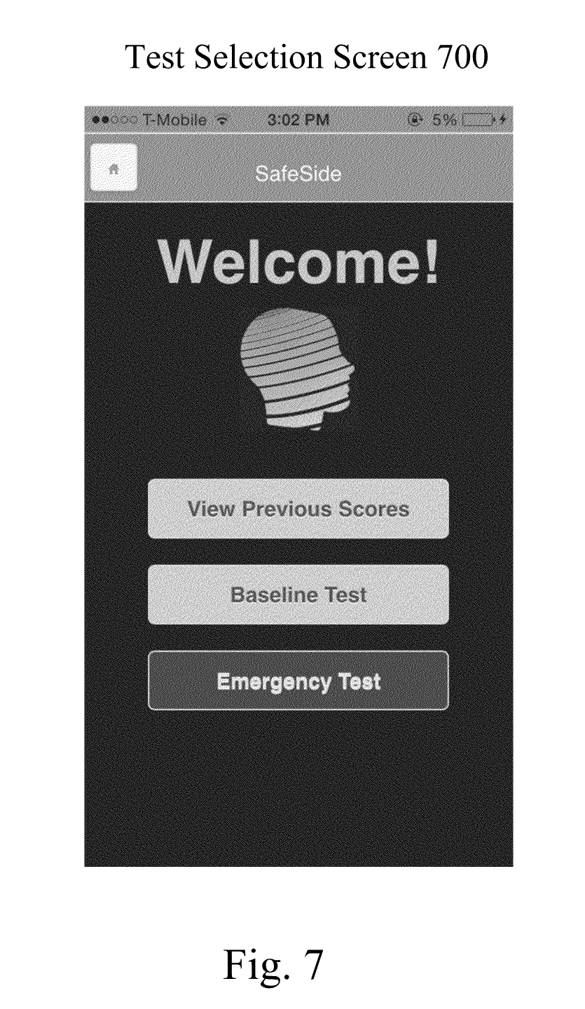

FIG. 7 is an illustrative screenshot of a test selection screen 700. Referring to FIGS. 2, 4, and 6, screen 700 may be displayed by user output 208 to allow a user to choose between beginning injury assessment process 400 or baseline measurement process 600, or reviewing previous test scores.

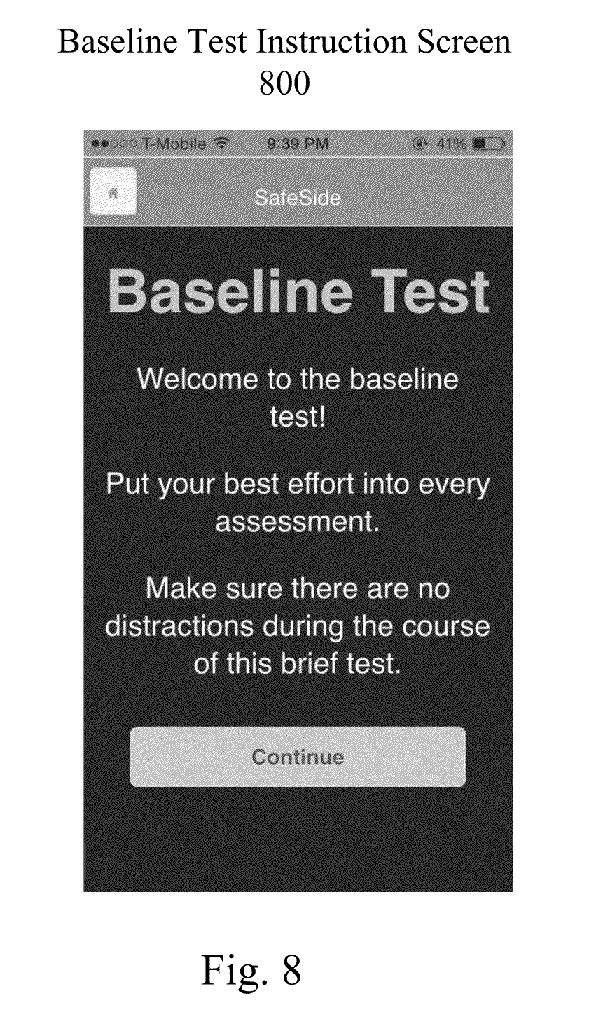

FIG. 8 is an illustrative screenshot of an baseline test instruction screen 800. Referring to FIGS. 2 and 6, screen 800 may be displayed by user output 208 during baseline measurement process 600. FIG. 8 informs a user that they are about to begin a baseline assessment test.

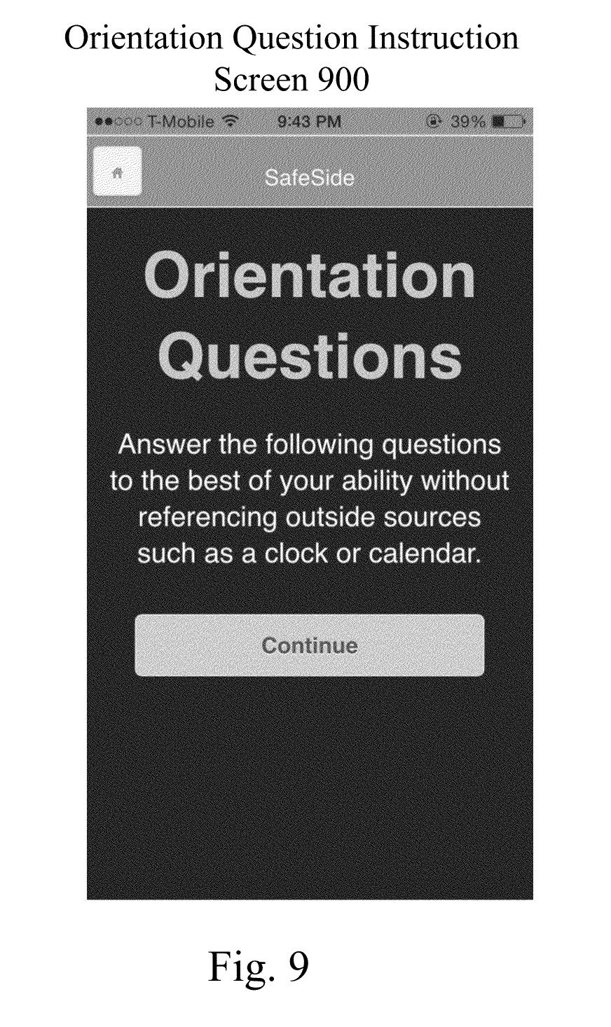

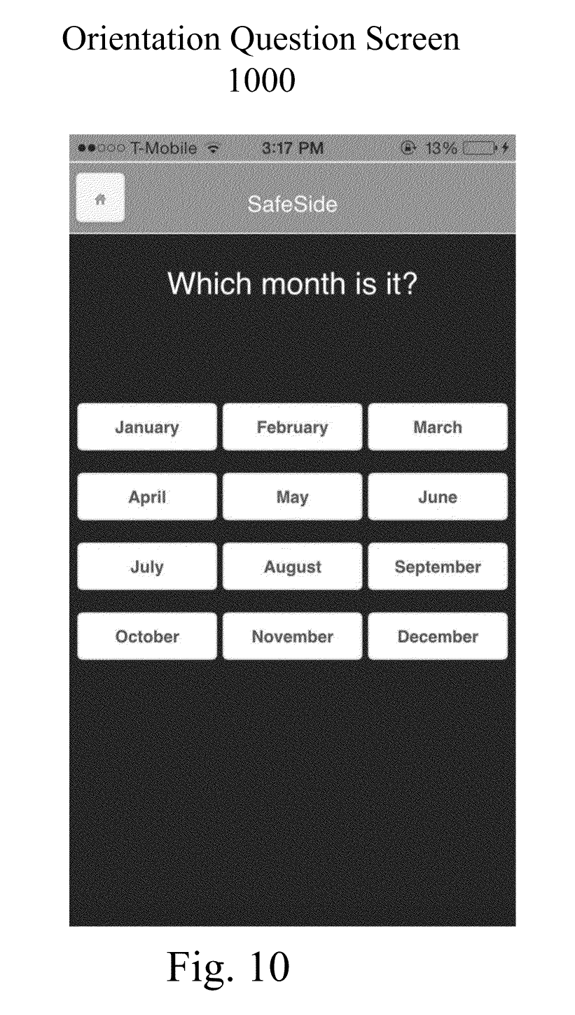

FIG. 9 is an illustrative screenshot of an orientation question instruction screen 900. Referring to FIGS. 2 and 4, screen 900 may be displayed by user output 208 during injury assessment process 400. FIG. 9 explains how the user is to respond to an orientation question such as is depicted in FIG. 10. As depicted, an orientation question may be related to the time.

FIG. 10 is an illustrative screenshot of an orientation question screen 1000. Referring to FIGS. 2 and 4, screen 1000 may be displayed by user output 208 during injury assessment process 400. As depicted, a user should respond to the depicted screen by selecting the current month. The score for such a question may be calculated based on whether the question was answered correctly and the time taken to answer. There may be alternative or additional orientation question screens 1000, such as asking the user to indicate the approximate time of day, the day of the week, the city in which the user is currently located, or other suitable questions.

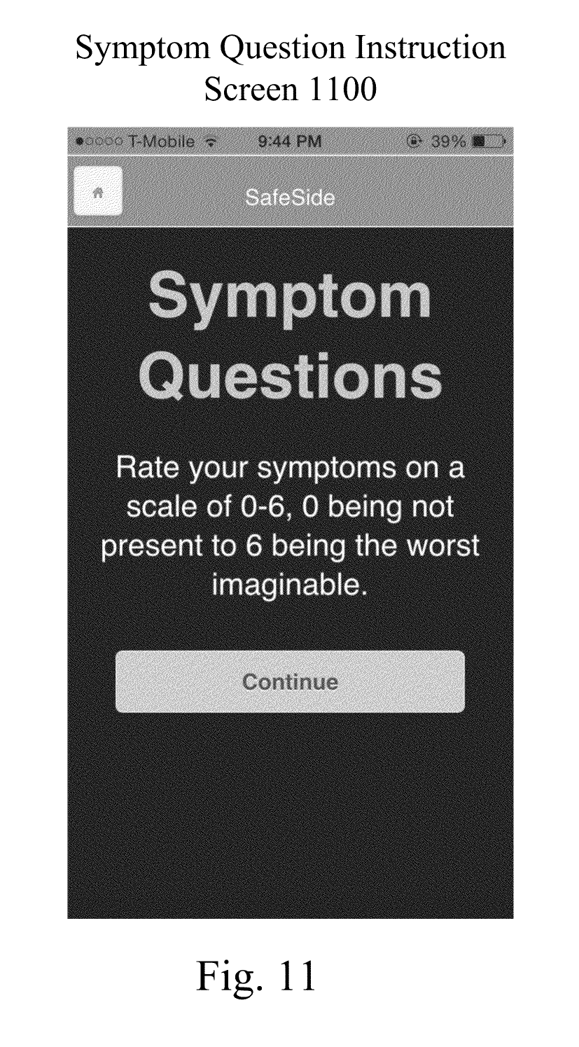

FIG. 11 is an illustrative of a symptom question instruction screen 1100. Referring to FIGS. 2, 4, and 6, screen 1100 may be displayed by user output 208 during injury assessment process 400 and baseline measurement process 600. As explained in screen 1100, a symptom question asks a user to rate a symptom on a numerical scale. In some implementations, one or more symptom questions may be yes/no questions, such as whether the user is experiencing ringing in the ears.

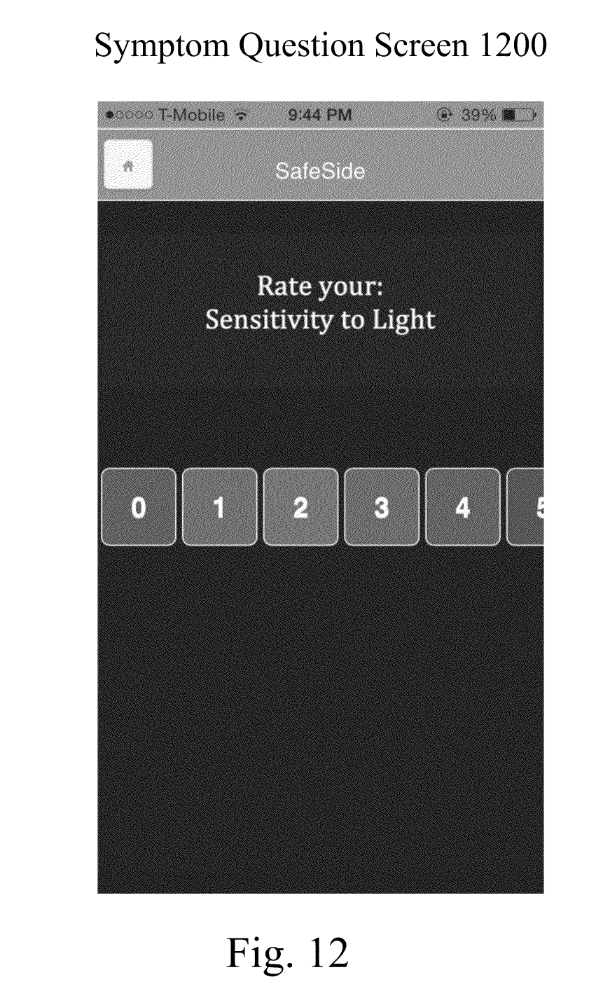

FIG. 12 is an illustrative screenshot of a symptom question screen 1200. Referring to FIGS. 2, 4, and 6, screen 1200 may be displayed by user output 208 during injury assessment process 400 and baseline measurement process 600. As depicted, a user should respond to the depicted screen by selecting their current sensitivity to light. The score for such a question may be calculated based on the sensitivity selected by the user. There may be alternative or additional orientation question screens 1200, such as asking the user how dizzy they feel, the extent of any pain they feel, or other suitable questions.

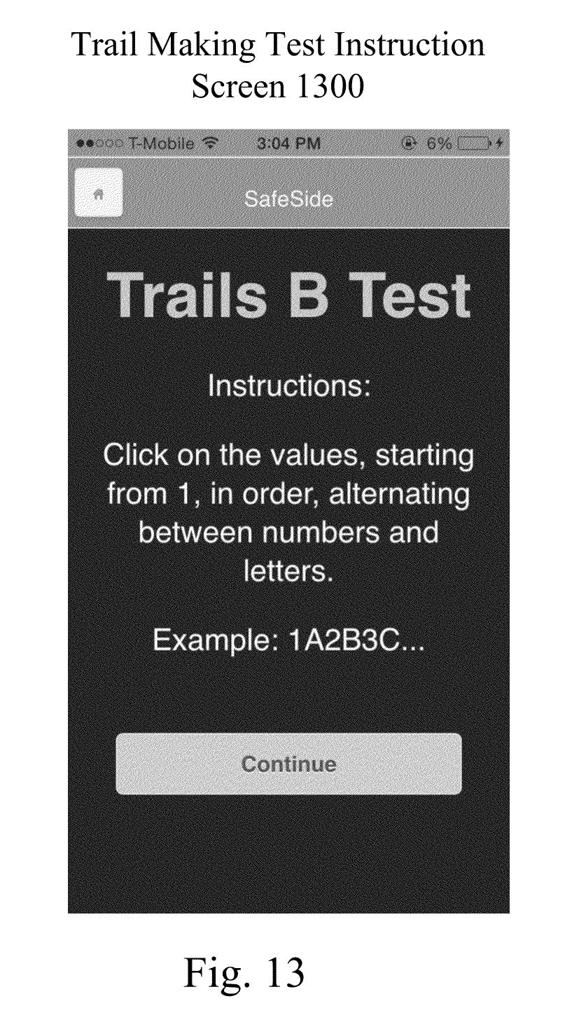

FIG. 13 is an illustrative screenshot of a Trail Making Test instruction screen 1300. Referring to FIGS. 2, 4, and 6, screen 1300 may be displayed by user output 208 during injury assessment process 400 and baseline measurement process 600. As explained in screen 1300, a Trail Making Test comprises displaying a grid of characters and requiring a user to select the characters in a prescribed order.

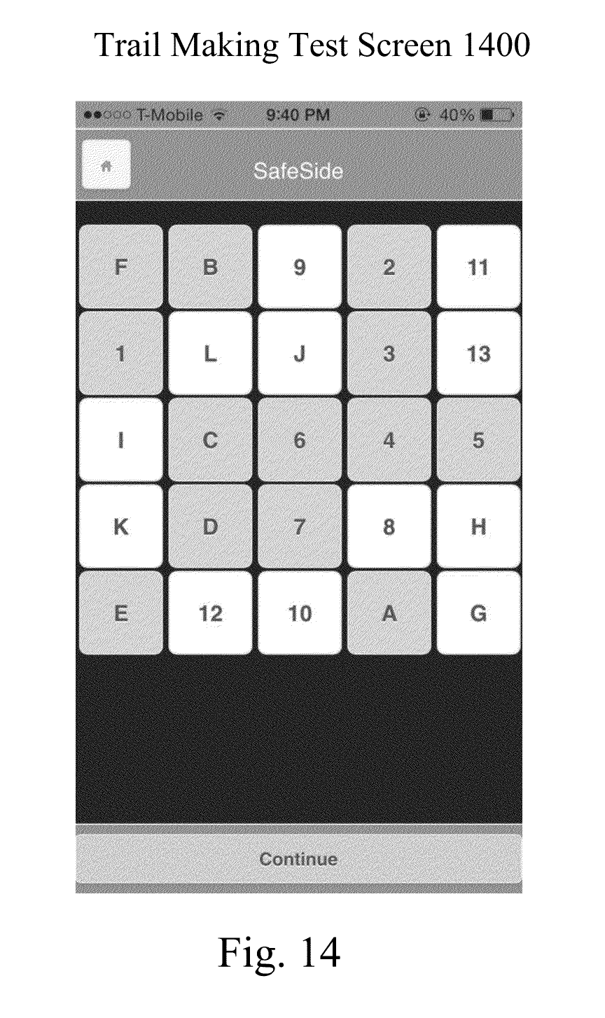

FIG. 14 is an illustrative screenshot of a Trail Making Test screen 1400. Referring to FIGS. 2, 4, and 6, screen 1400 may be displayed by user output 208 during injury assessment process 400 and baseline measurement process 600. As depicted, were screen 1400 to appear immediately after screen 1300, a user should respond to the depicted screen by selecting the number 1, followed by the letter A, followed by the number 2, and so on. The score for such a question may be calculated based on the number of correct entries selected during a predetermined period of time, on the time taken to properly complete the task, or on some other suitable criteria.

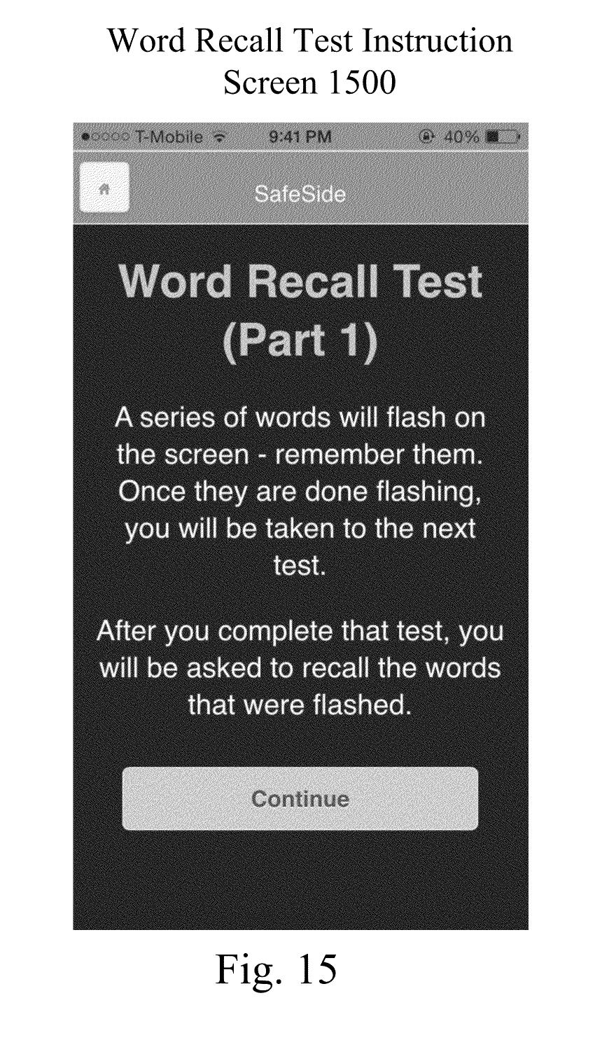

FIG. 15 is an illustrative screenshot of a first word recall test instruction screen 1500. Referring to FIGS. 2, 4, and 6, screen 1500 may be displayed by user output 208 during injury assessment process 400 and baseline measurement process 600. As explained in screen 1500, a word recall test comprises displaying a series of words, and later displaying one or more words and asking the user whether the later-displayed words were in the earlier series of words.

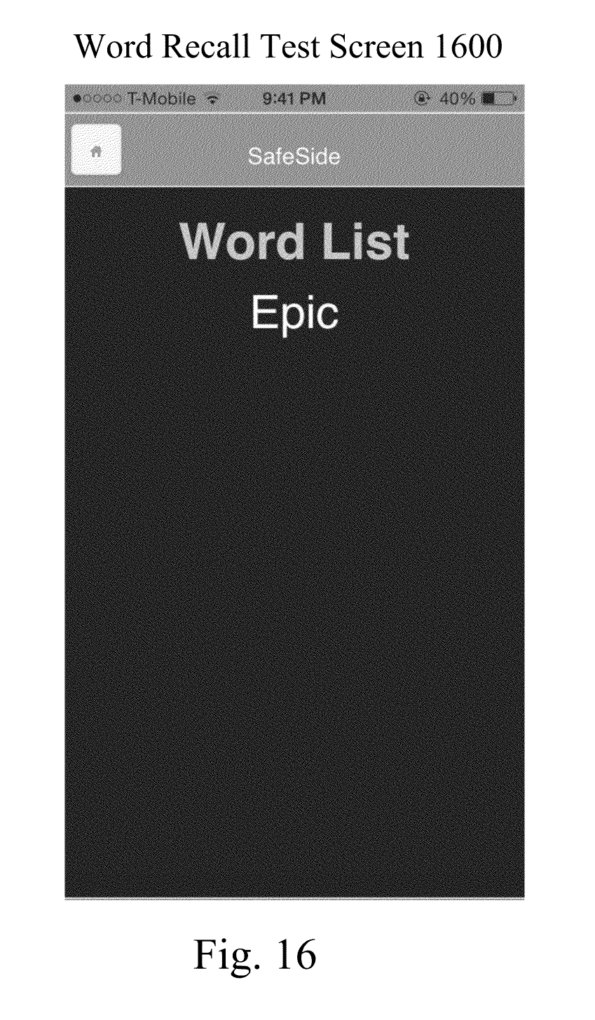

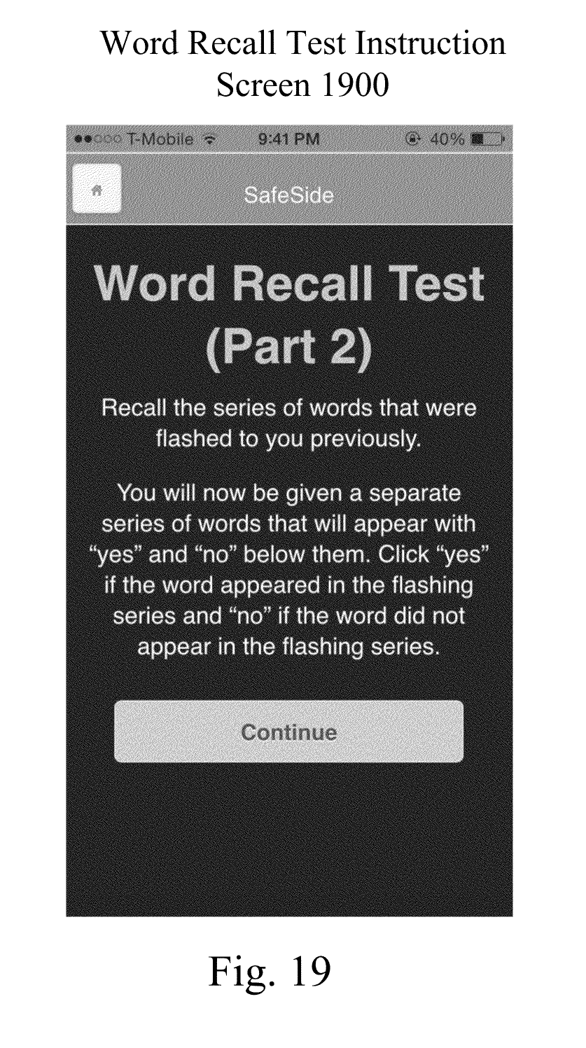

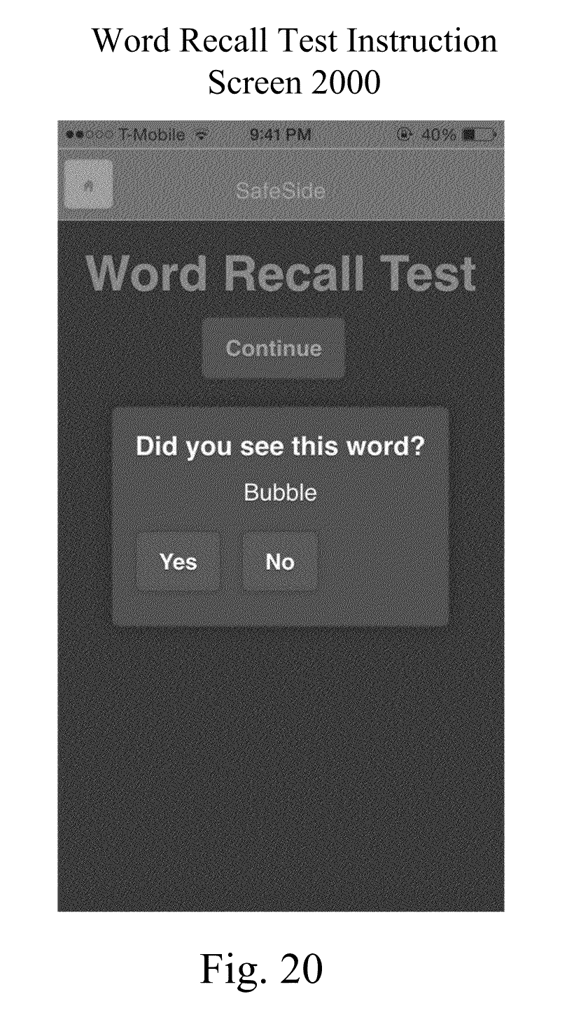

FIG. 16 is an illustrative screenshot of a first word recall test screen 1600. Referring to FIGS. 2, 4, and 6, screen 1600 may be displayed by user output 208 during injury assessment process 400 and baseline measurement process 600. As explained in reference to FIG. 15, a list of words will be displayed; as depicted, a first word in the list is being displayed, and will later be replaced with another word in the list. The user does not need to respond to the word list displayed in screen 1600, but instead remember the words for later questions, as described in relation to FIGS. 19 and 20.

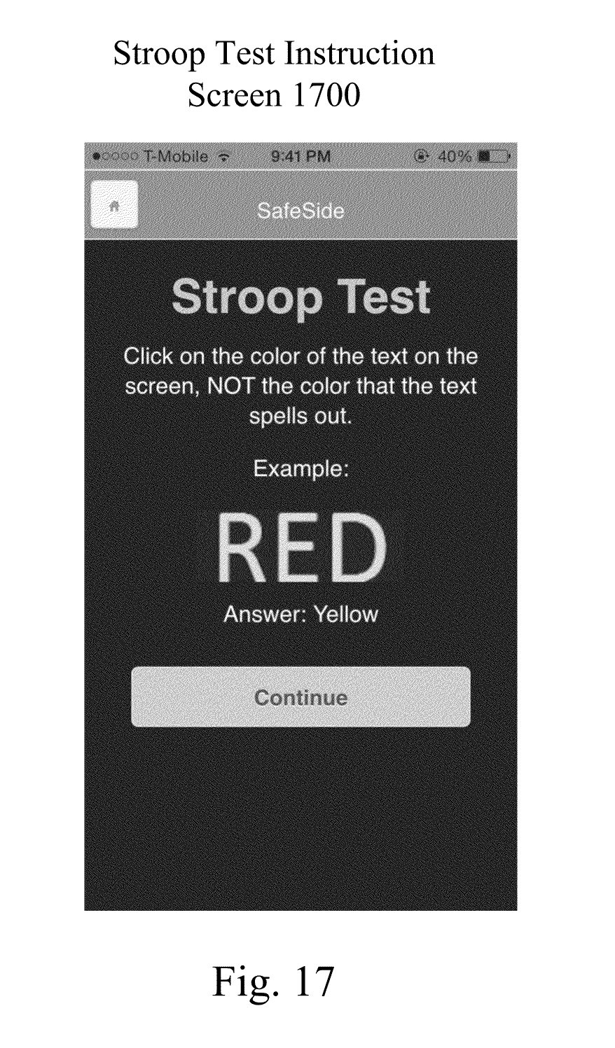

FIG. 17 is an illustrative screenshot of a Stroop test instruction screen 1700. Referring to FIGS. 2, 4, and 6, screen 1700 may be displayed by user output 208 during injury assessment process 400 and baseline measurement process 600. As explained in screen 1700, a Stroop test comprises displaying a color word in a color that may not match the color word, and requiring the user to identify the color in which the word is displayed.

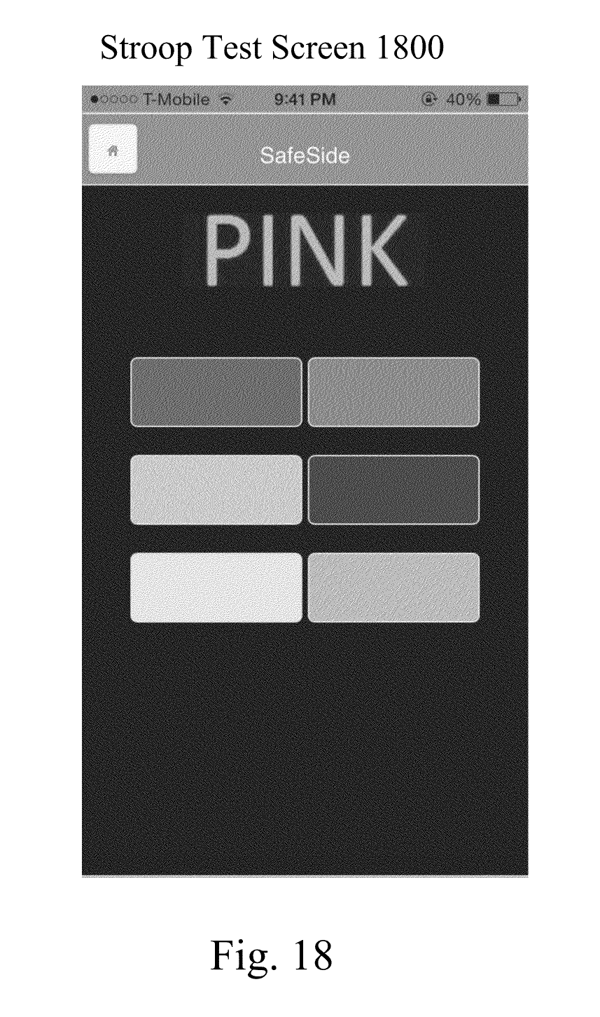

FIG. 18 is an illustrative screenshot of a Stroop test screen 1800. Referring to FIGS. 2, 4, and 6, screen 1800 may be displayed by user output 208 during injury assessment process 400 and baseline measurement process 600. As explained in reference to FIG. 17, a user should respond to the depicted screen by selecting the color in which the word “pink” is displayed rather than the color pink itself. The score for such a question may be calculated based on whether the question was answered correctly and the time taken to answer.

FIG. 19 is an illustrative screenshot of a second word recall test instruction screen 1900. Referring to FIGS. 2, 4, and 6, screen 1900 may be displayed by user output 208 during injury assessment process 400 and baseline measurement process 600. As explained in screen 1500, screen 1900 appears after screen 1600, and may appear after one or more other questions have been administered to the user. Screen 1900 explains that a user is to answer whether a displayed word was included in an earlier list of words.

FIG. 20 is an illustrative screenshot of a second word recall test screen 2000. Referring to FIGS. 2, 4, and 6, screen 2000 may be displayed by user output 208 during injury assessment process 400 and baseline measurement process 600. As explained in reference to FIGS. 15 and 19, a user should respond to the depicted screen 2000 by selecting “yes” if the word “bubble” appeared in an earlier displayed word list, and “no” if not. The score for such a question may be calculated based on whether the question was answered correctly and the time taken to answer.

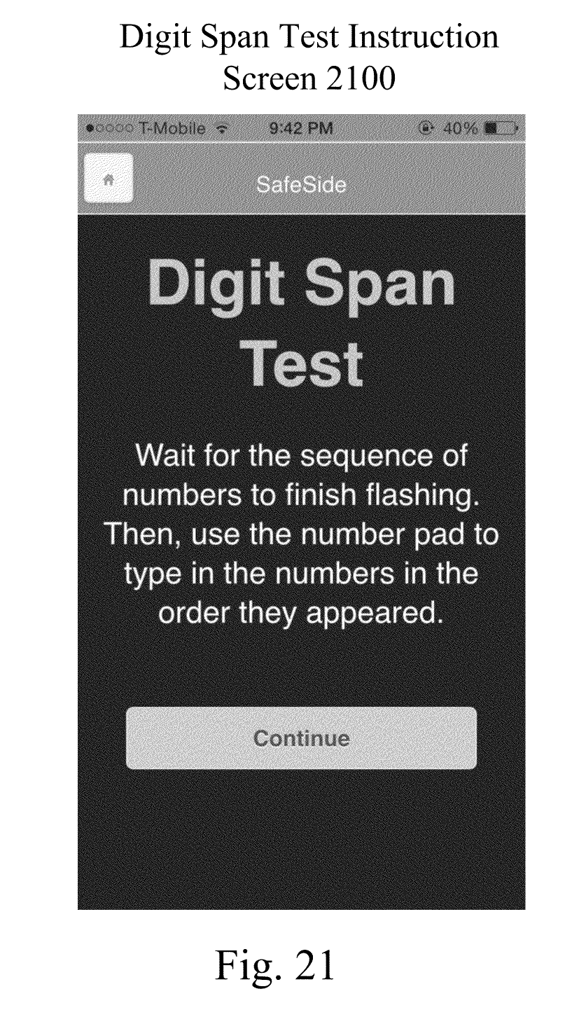

FIG. 21 is an illustrative screenshot of a digit span test instruction screen 2100. Referring to FIGS. 2, 4, and 6, screen 2100 may be displayed by user output 208 during injury assessment process 400 and baseline measurement process 600. As explained in screen 2100, a digit span test comprises displaying a series of numbers and requiring the user to then repeat the series of numbers in the same order as they were displayed.

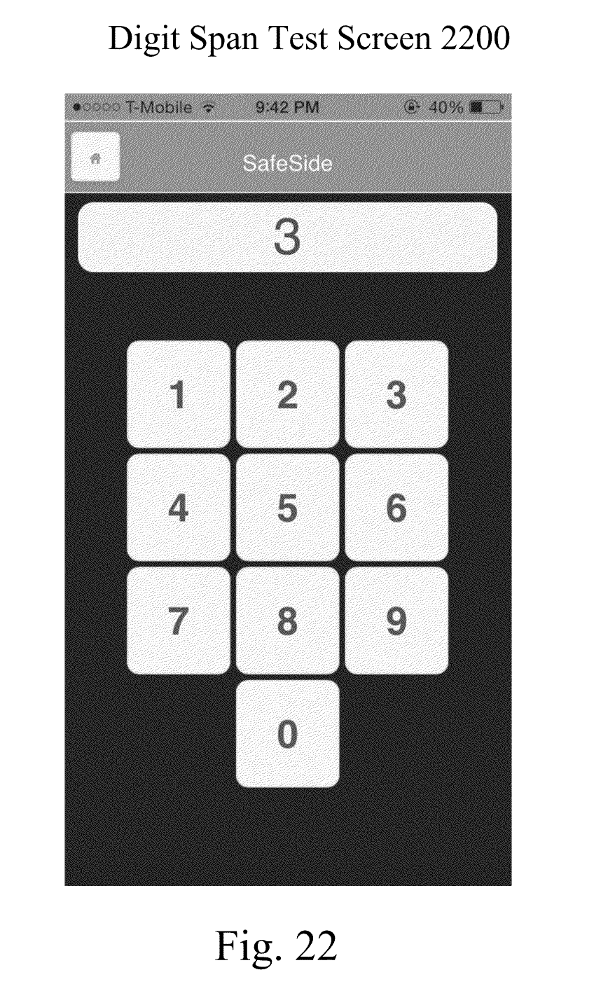

FIG. 22 is an illustrative screenshot of a digit span test screen 2200. Referring to FIGS. 2, 4, and 6, screen 2200 may be displayed by user output 208 during injury assessment process 400 and baseline measurement process 600. As explained in reference to FIG. 21, a user should respond to the depicted screen by entering a series of numbers in the same order that an earlier series of numbers were displayed. The score for such a question may be calculated based on whether the question was answered correctly and the time taken to answer.

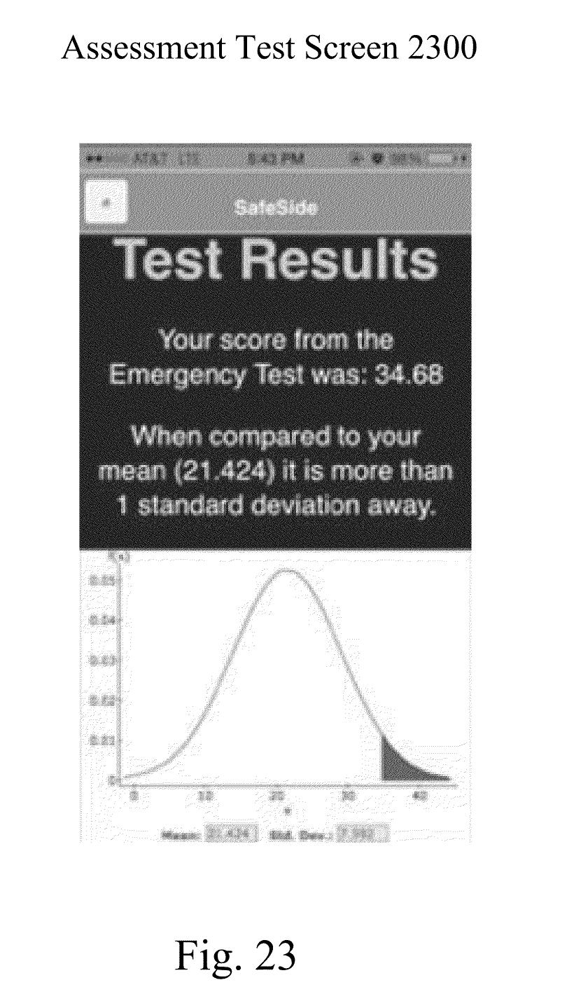

FIG. 23 is an illustrative screenshot of an assessment test screen 2300. Referring to FIGS. 2 and 4, screen 2300 may be displayed by user output 208 during injury assessment process 400. Screen 2300 compares the results of an assessment test as compared to the user's historical baseline test results.

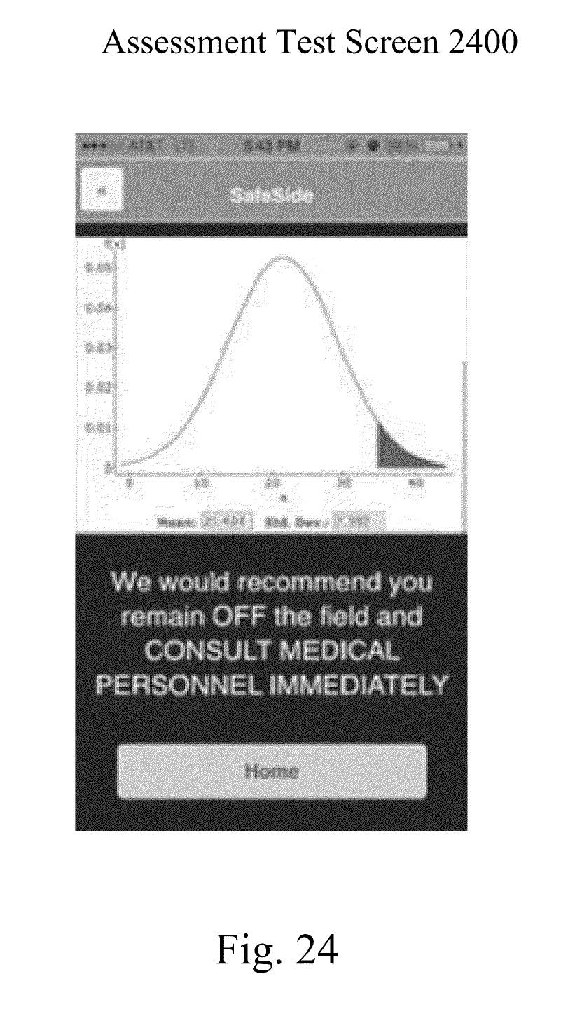

FIG. 24 is an illustrative screenshot of an assessment test screen 2400. Referring to FIGS. 2 and 4, screen 2400 may be displayed by user output 208 during injury assessment process 400. Screen 2400 compares the results of an assessment test as compared to the user's historical baseline test results. As depicted, a higher score indicates greater likelihood of impairment, and as the depicted test results are greater than a threshold, screen 2400 suggests that the user seek medical attention.

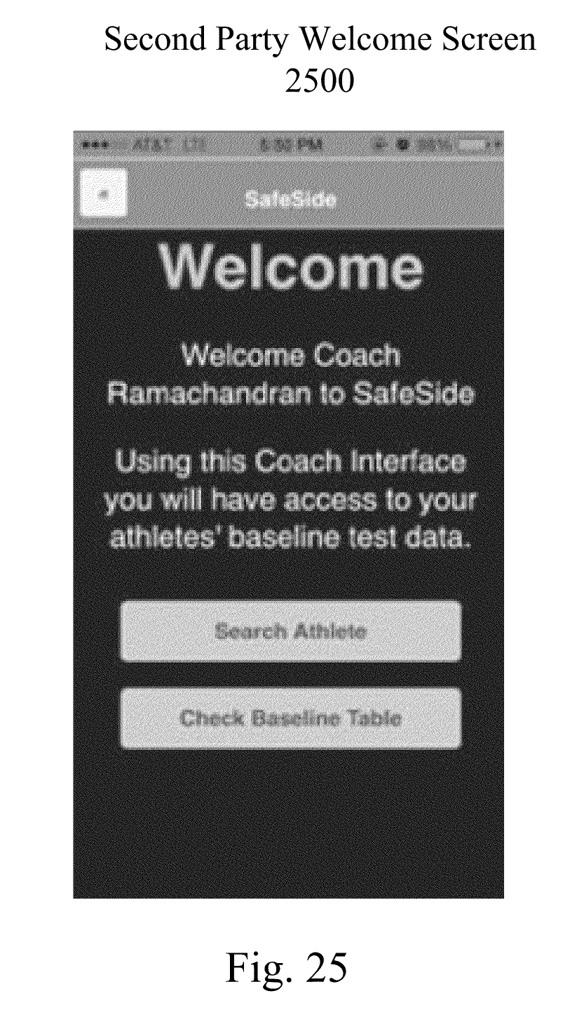

FIG. 25 is an illustrative screenshot of a second party welcome screen 2500. Referring to FIG. 2, screen 2500 may be displayed by a user output 208 of a device authorized to display another user's baseline test results. As depicted, screen 2500 allows a coach to choose whether to view a specific user's results, or to view a table of all users whose results the coach is authorized to view. Screen 2500 may be displayed on a coach's user device 200, a medical professional's user device 200, or some other suitable person's user device 200.

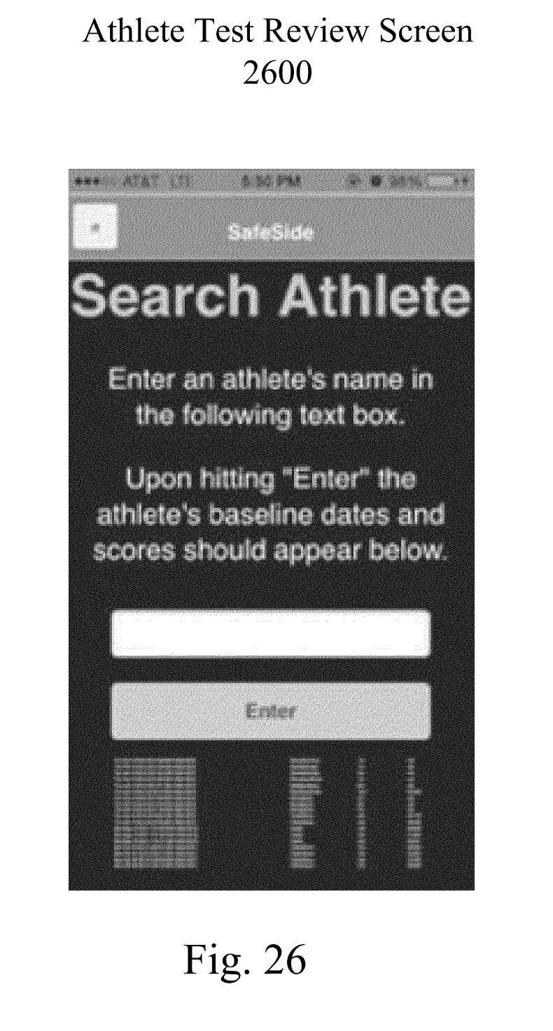

FIG. 26 is an illustrative screenshot of an athlete test review screen 2600. Referring to FIG. 2, screen 2600 may be displayed by a user output 208 of a device authorized to display a user's baseline test results. As depicted, screen 2600 allows a first user (e.g., a coach or a parent) to enter the name of a second user (e.g., an athlete). If the first user is authorized to view the second user's results, screen 2600 will display the second user's results. Screen 2600 may be displayed on a coach's user device 200, a medical professional's user device 200, or some other suitable person's user device 200.

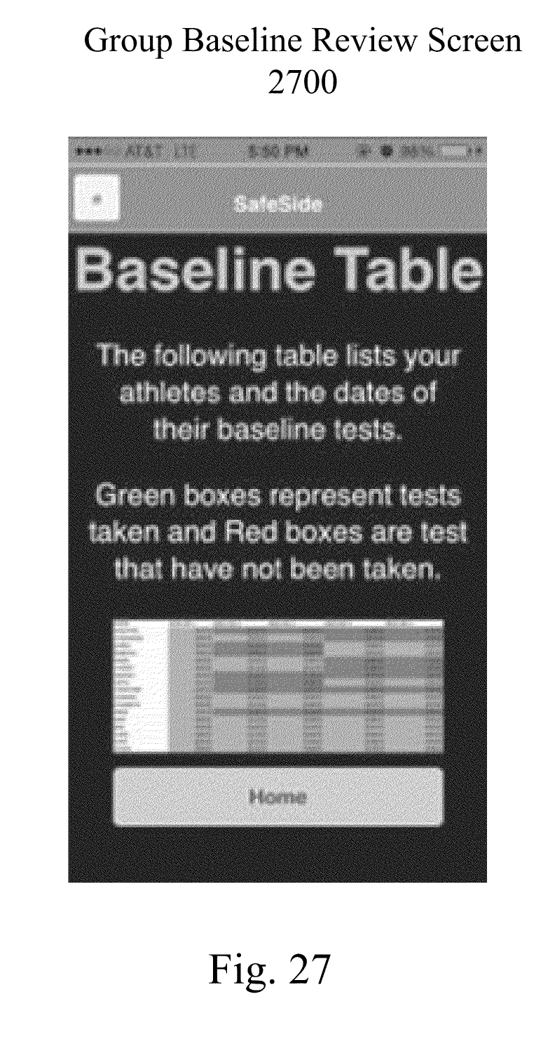

FIG. 27 is an illustrative screenshot of a group baseline review screen 2700. Referring to FIG. 2, screen 2700 may be displayed by a user output 208 of a device authorized to display a user's baseline test results. As depicted, screen 2700 displays results associated with a list of athletes to a user authorized to view the athletes' test results. Screen 2700 may be displayed on a coach's user device 200, a medical professional's user device 200, or some other suitable person's user device 200.

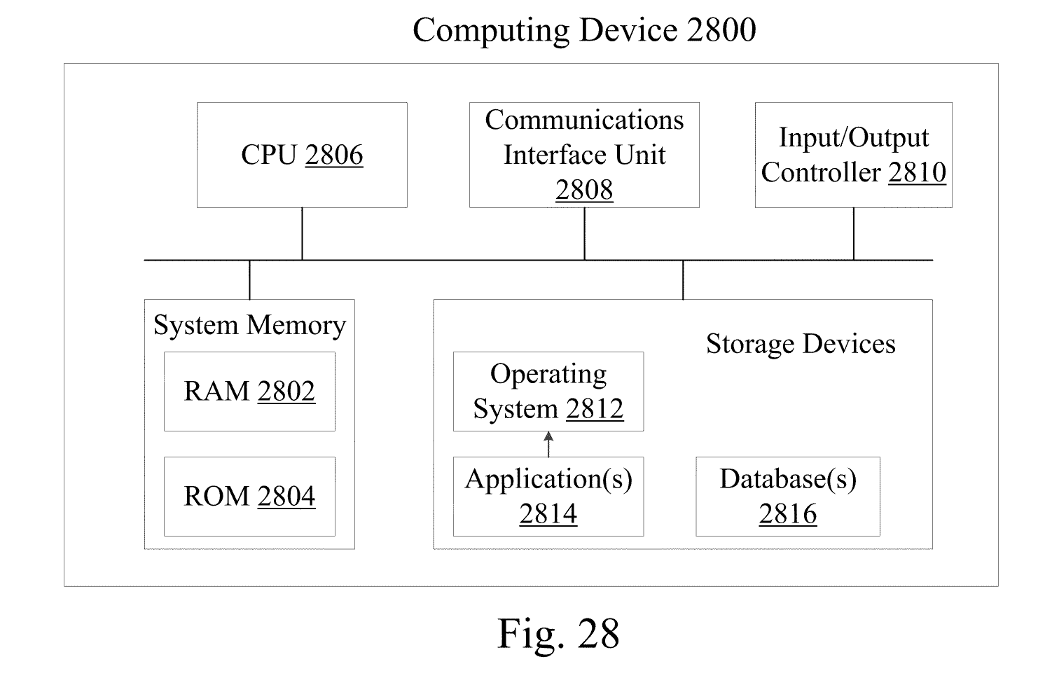

FIG. 28 is a block diagram of a computing device that can be used to implement or support any of the components of the system of FIG. 1, 2, or 3, and for performing any of the processes described herein. Assessment server 300 may be implemented on one or more computing devices 2800 having suitable circuitry, and user device 102 may communicate with assessment server 106 through one or more computing devices 2800 having suitable circuitry. In certain aspects, a plurality of the components of injury assessment system 100 may be included within one computing device 2800. In certain implementations, a component of injury assessment system 100 may be implemented across several computing devices 2800.

The computing device 2800 comprises at least one communications interface unit, an input/output controller 2810, system memory, and one or more data storage devices. This can support a network connection, such as a connection to network 104 in FIG. 2. The system memory includes at least one random access memory (RAM 2802) and at least one read-only memory (ROM 2804). RAM 2802 can support the user database 2810 of FIG. 2, for example. All of these elements are in communication with a central processing unit (CPU 2806) to facilitate the operation of the computing device 2800. The computing device 2800 may be configured in many different ways. For example, the computing device 2800 may be a conventional standalone computer or, alternatively, the functions of computing device 2800 may be distributed across multiple computer systems and architectures. In FIG. 28, the computing device 2800 may be linked, via network or local network, to other servers or systems.

The computing device 2800 may be configured in a distributed architecture, wherein databases and processors are housed in separate units or locations. Some units perform primary processing functions and contain, at a minimum, a general controller or a processor and a system memory. In distributed architecture implementations, each of these units may be attached via the communications interface unit 2808 to a communications hub or port (not shown) that serves as a primary communication link with other servers, client or user computers, and other related devices. The communications hub or port may have minimal processing capability itself, serving primarily as a communications router. A variety of communications protocols may be part of the system, including, but not limited to: Ethernet, SAP, SAS™, ATP, BLUETOOTH™, GSM, and TCP/IP.

The CPU 2806 comprises a processor, such as one or more conventional microprocessors and one or more supplementary co-processors such as math co-processors for offloading workload from the CPU 2806. The CPU 2806 is in communication with the communications interface unit 2808 and the input/output controller 2810, through which the CPU 2806 communicates with other devices such as other servers, user terminals, or devices. The communications interface unit 2808 and the input/output controller 2810 may include multiple communication channels for simultaneous communication with, for example, other processors, servers, or client terminals.

The CPU 2806 is also in communication with the data storage device. The data storage device may comprise an appropriate combination of magnetic, optical, or semiconductor memory, and may include, for example, RAM 2802, ROM 2804, flash drive, an optical disc such as a compact disc, or a hard disk or drive. The CPU 2806 and the data storage device each may be, for example, located entirely within a single computer or other computing device; or connected to each other by a communication medium, such as a USB port, serial port cable, a coaxial cable, an Ethernet cable, a telephone line, a radio frequency transceiver, or other similar wireless or wired medium or combination of the foregoing. For example, the CPU 2806 may be connected to the data storage device via the communications interface unit 2808. The CPU 2806 may be configured to perform one or more particular processing functions.

The data storage device may store, for example, (i) an operating system 2812 for the computing device 2800; (ii) one or more applications 2814 (e.g., computer program code or a computer program product) adapted to direct the CPU 2806 in accordance with the systems and methods described here, and particularly in accordance with the processes described in detail with regard to the CPU 2806; or (iii) database(s) 2816 adapted to store information that may be utilized to store information required by the program. The depicted database 2816 can be any suitable database system, and can be a local or distributed database system.

The operating system 2812 and applications 2814 may be stored, for example, in a compressed, an uncompiled and an encrypted format, and may include computer program code. The instructions of the program may be read into a main memory of the processor from a computer-readable medium other than the data storage device, such as from the ROM 2804 or from the RAM 2802, or from a computer data signal embodied in a carrier wave, such as that found within the well-known Web pages transferred among devices connected to the Internet. While execution of sequences of instructions in the program causes the CPU 2806 to perform the process steps described herein, hard-wired circuitry may be used in place of, or in combination with, software instructions for implementation of the processes of the present disclosure. Thus, the systems and methods described are not limited to any specific combination of hardware and software.

Suitable computer program code may be provided for performing one or more functions in relation to an assessment system as described herein. The program also may include program elements such as an operating system 2812, a database management system, and “device drivers” that allow the processor to interface with computer peripheral devices (e.g., a video display, a keyboard, a computer mouse, etc.) via the input/output controller 2810.

The term “computer-readable medium” as used herein refers to any non-transitory medium that provides or participates in providing instructions to the processor of the computing device 2800 (or any other processor of a device described herein) for execution. Such a medium may take many forms, including but not limited to, non-volatile media and volatile media. Non-volatile media include, for example, optical, magnetic, or opto-magnetic disks, or integrated circuit memory, such as flash memory. Volatile media include dynamic random access memory (DRAM), which typically constitutes the main memory. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM or EEPROM (electronically erasable programmable read-only memory), a FLASH-EEPROM, any other memory chip or cartridge, or any other non-transitory medium from which a computer can read.

Various forms of computer readable media may be involved in carrying one or more sequences of one or more instructions to the CPU 2806 (or any other processor of a device described herein) for execution. For example, the instructions may initially be borne on a magnetic disk of a remote computer (not shown). The remote computer can load the instructions into its dynamic memory and send the instructions over an Ethernet connection, cable line, or even telephone line using a modem. A communications device local to a computing device 2800 (e.g., a server) can receive the data on the respective communications line and place the data on a system bus for the processor. The system bus carries the data to main memory, from which the processor retrieves and executes the instructions. The instructions received by main memory may optionally be stored in memory either before or after execution by the processor. In addition, instructions may be received via a communication port as electrical, electromagnetic, or optical signals, which are exemplary forms of wireless communications or data streams that carry various types of information.

Some implementations of the above described may be implemented by the preparation of application-specific integrated circuits or by interconnecting an appropriate network of conventional component circuits, as will be apparent to those skilled in the art. Those of skill in the art would understand that information and signals may be represented using any of a variety of different technologies and techniques. For example, data, instructions, requests, information, signals, bits, symbols, and chips that may be referenced throughout the above description may be represented by voltages, currents, electromagnetic waves, magnetic fields or particles, optical fields or particles, or any combination thereof.

While various embodiments of the present disclosure have been shown and described herein, it will be obvious to those skilled in the art that such embodiments are provided by way of example only. Numerous variations, changes, and substitutions will now occur to those skilled in the art without departing from the disclosure. For example, assessment system 100 may be implemented entirely on a user device 102, which may eliminate the need for an assessment server 106; users subject to tests may have an athlete-specific user device 200 distinct from other users' devices; a user device 200 may display different information and options depending on whether a user signing in is a tested user or is authorized to view other users' information; a user device 200 may offer an option to contact a medical professional in response to a poor assessment test; a user device 200 may generate an alert if a user's baseline tests show a pattern of decline; users may select to view information regarding concussions; alternate or additional baseline or assessment tasks may be provided, such as a fatigue scale, a mood scale, a two-choice reaction time test, a four-choice reaction time test, a procedural reaction time test, a running memory continuous performance task, a mathematical processing task, a digit set comparison task, a logical reasoning task, a Tower of Hanoi task, a spatial processing task, and a tracking task; a user's assessment tasks may be based on the baseline tasks they have most recently completed; system 100 may have access to scheduling information, and may inform other users if an athlete has not taken a baseline exam within a predetermined period of the beginning of a scheduled practice or game; performance thresholds may be based in part on test results of populations similar to the user, standardized thresholds, or clinically calibrated thresholds associated with injury risk. It should be understood that various alternatives to the embodiments of the disclosure described herein may be employed in practicing the disclosure. Elements of an implementation of the systems and methods described herein may be independently implemented or combined with other implementations. It is intended that the following claims define the scope of the disclosure and that methods and structures within the scope of these claims and their equivalents be covered thereby.

1. A system for assessing a possible brain injury in a human, the system comprising:
a processor operably connected to a user database configured to store a performance threshold associated with a first user, and configured to:
present one or more assessment tasks to the first user,
receive input from the first user in response to the one or more assessment tasks, and
output an indicator representative of a comparison of an input score to the performance threshold.

2. The system of claim 1, wherein the processor is further configured to:
determine whether the first user has completed a baseline test within a predetermined period of time, wherein a baseline test comprises one or more baseline tasks;
if the first user has not completed a baseline test within the predetermined period of time,
output a prompt to request that the first user complete a baseline test; and
administer a first baseline test comprising one or more baseline tasks,
wherein the performance threshold is based on input received from the first user in response to the first baseline test.

3-4. (canceled)

5. The system of claim 2, wherein the processor is further configured to randomly generate at least one of the one or more baseline tasks, wherein the one or more baseline tasks include at least one of a Stroop test, a digit span test, a Trail Making Test, answering a symptom question, answering an orientation question, a word recall test, and a balance test.

6-10. (canceled)

11. The system of claim 2, wherein the processor is further configured to administer the baseline test within one hour of user exercise in an absence of a possible brain injury.

12. The system of claim 2, wherein the processor is further configured to:
administer a second baseline test to the first user, and
identify a change in performance from the first baseline test to the second baseline test.

13. The system of claim 12, wherein the processor is further configured to modify the performance threshold based on the change in performance from the first baseline test to the second baseline test.

14. The system of claim 12, wherein the processor is further configured to administer the second baseline test to the first user after identifying a possible brain injury.

15. The system of claim 1, wherein the processor is further configured to output an indicator representative of the comparison to a second user.

16-19. (canceled)

20. The system of claim 15, wherein the processor is further configured to:
output an indicator representative of the comparison to a third user,
receive feedback from the third user responding to the indicator representative of the comparison, and
output the third-user feedback to the second user, wherein the second user is at least one of a coach, a guardian, a referee, and a medical professional.

21. The system of claim 1, wherein the processor is further configured to present the one or more assessment tasks for completion within a predetermined period of response time.

22. (canceled)

23. The system of claim 1, wherein the one or more assessment tasks include a Stroop test.

24. The system of claim 23, wherein the processor is further configured to:
identify whether the first user is color-blind; and
identify Stroop test questions unaffected by a color-blindness of the first user.

25. (canceled)

26. The system of claim 1, wherein the one or more assessment tasks include at least one of a digit span test, a Trail Making Test, answering a symptom question, answering an orientation question, a word recall test, and a balance test.

27-29. (canceled)

30. The system of claim 1, wherein the processor is further configured to receive sensor data associated with the user, wherein the input score is based on the sensor data.

31-32. (canceled)

33. The system of claim 1, wherein the possible brain injury is a possible concussion.

34. The system of claim 1, wherein the one or more assessment tasks include one or more gating tasks associated with severe impairment, and wherein the processor is further configured to:
present the one or more gating tasks to the first user,
receive input in response to the one or more gating tasks, and
if the input received in response to the one or more gating tasks is indicative of severe impairment, stop administration of assessment tasks and output a warning.

35. A computer-implemented method for assessing a possible brain injury in a human, comprising:
presenting one or more assessment tasks to a first user,
receiving input from the first user in response to the one or more assessment tasks; and
outputting an indicator representative of a comparison of an input score to a performance threshold associated with the first user.

36. The method of claim 35, further comprising:
determining whether the first user has completed a baseline test within a predetermined period of time, wherein a baseline test comprises one or more baseline tasks;
if the first user has not completed a baseline test within the predetermined period of time,
outputting a prompt to request that the first user complete a baseline test; and
administering a first baseline test comprising one or more baseline tasks to the first user,
wherein the performance threshold is based on input received from the first user in response to the first baseline test.

37-38. (canceled)

39. The method of claim 36, further comprising randomly generating at least one of the one or more baseline tasks, wherein the one or more baseline tasks include at least one of a Stroop test, a digit span test, a Trail Making Test, answering a symptom question, answering an orientation question, a word recall test, and a balance test.

40-44. (canceled)

45. The method of claim 36, wherein the baseline test is administered within one hour of user exercise in an absence of a possible brain injury.

46. The method of claim 36, further comprising:
administering a second baseline test to the first user, and
identifying a change in performance from the first baseline test to the second baseline test.

47. The method of claim 46, further comprising modifying the performance threshold based on the change in performance from the first baseline test to the second baseline test.

48. The method of claim 46, further comprising administering the second baseline test to the first user after identifying a possible brain injury.

49. The method of claim 35, further comprising outputting an indicator representative of the comparison to a second user.

50-53. (canceled)

54. The method of claim 49, further comprising:
outputting an indicator representative of the comparison to a third user,
receiving feedback from the third user responding to the indicator representative of the comparison; and
outputting the third-user feedback to the second user, wherein the second user is at least one of a coach, a guardian, a referee, and a medical professional.

55. The method of claim 35, further comprising presenting the one or more assessment tasks for completion within a predetermined period of response time.

56. (canceled)

57. The method of claim 35, wherein the one or more assessment tasks include a Stroop test.

58. The method of claim 57, further comprising:
identifying whether the first user is color-blind; and
identifying Stroop test questions unaffected by a color-blindness of the first user.

59. (canceled)

60. The method of claim 35, wherein the one or more assessment tasks include at least one of a digit span test, a Trail Making Test, answering a symptom question, answering an orientation question, a word recall test, and a balance test.

61-63. (canceled)

64. The method of claim 35, further comprising receiving sensor data associated with the user, wherein the input score is based on the sensor data.

65-66. (canceled)

67. The method of claim 35, wherein the possible brain injury is a possible concussion.

68. The method of claim 35, wherein the one or more assessment tasks include one or more gating tasks associated with severe impairment, and the method further comprises:
presenting the one or more gating tasks to the first user,
receiving input in response to the one or more gating tasks, and
if the input received in response to the one or more gating tasks is indicative of severe impairment, stopping administration of assessment tasks and outputting a warning.
